(19) United States
(12) Patent Application Publication
(10) Pub. No.: US 2021/0347736 A1
(43) Pub. Date: Nov. 11, 2021

(54) CRYSTALLINE FORMS OF A FARNESOID X RECEPTOR AGONIST

(71) Applicant: Metacrine, Inc., San Diego, CA (US)

(72) Inventors: Nicholas D. SMITH, San Diego, CA (US); Robert MANSFIELD, San Diego, CA (US)

(21) Appl. No.: 17/276,763

(22) PCT Filed: Sep. 17, 2019
(86) PCT No.: PCT/US19/51605

§ 371 Date: Mar. 16, 2021

Related U.S. Application Data
(60) Provisional application No. 62/733,007, filed on Sep. 18, 2018.

(51) Int. Cl. C07D 231/12; A61K 9/16

(52) U.S. Cl. CPC C07D 231/12; C07B 2200/13; A61K 9/1635

(57) ABSTRACT

Described herein is the farnesoid X receptor agonist, trans-N-(3-(1-cyclopropyl-1H-pyrazol-4-yl)phenyl)-4-hydroxy-N-((trans-4-(4-methoxy-3-methyl phenyl)cyclohexyl)methyl)cyclohexane-carboxamide, including crystalline forms and pharmaceutically acceptable salts, solvates, and formulations thereof.

### CROSS-REFERENCE

This application claims benefit of U.S. Provisional Application No. 62/733,007, filed on Sep. 18, 2018 which is herein incorporated by reference in its entirety.

### FIELD OF THE INVENTION

Described herein are compounds that are farnesoid X receptor agonists, methods of making such compounds, pharmaceutical compositions and medicaments comprising such compounds, and methods of using such compounds in the treatment of conditions, diseases, or disorders associated with farnesoid X receptor activity.

### BACKGROUND OF THE INVENTION

Farnesoid X receptor (FXR) is a nuclear receptor highly expressed in the liver, intestine, kidney, adrenal glands, and adipose tissue. FXR regulates a wide variety of target genes involved in the control of bile acid synthesis and transport, lipid metabolism, and glucose homeostasis. FXR agonism is a treatment modality for many metabolic disorders, liver diseases or conditions, inflammatory conditions, gastrointestinal diseases, or cell proliferation diseases.

### SUMMARY OF THE INVENTION

Described herein is the farnesoid X receptor agonist, trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, including pharmaceutically acceptable solvates (including hydrates), polymorphs, and amorphous phases, and methods of uses thereof. trans-N-(3-(1-Cyclopropyl-1H-pyrazol-4-yl)phenyl)-4-hydroxy-N-((trans-4-(4-methoxy-3-methylphenyl)cyclohexyl)methyl)cyclohexanecarboxamide, as well as the pharmaceutically acceptable solvates (including hydrates), polymorphs, and amorphous phases thereof, are used in the manufacture of medicaments for the treatment of diseases or conditions in a mammal that would benefit from treatment with an FXR agonist.

Also described herein are methods for preparing crystalline forms of trans-N-(3-(1-cyclopropyl-1H-pyrazol-4-yl)phenyl)-4-hydroxy-N-((trans-4-(4-methoxy-3-methylphenyl)-cyclohexyl)methyl)cyclohexanecarboxamide. Further described are pharmaceutical compositions that include the crystalline forms and methods of using the FXR agonist in the treatment of diseases or conditions.

In one embodiment is a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof.

In another embodiment, the crystalline form of claim 1, wherein the trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide is a free base.

In another embodiment, described herein is a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide that has at least one of the following properties:
(a) an X-ray powder diffraction (XRPD) pattern substantially the same as shown in FIG. 1;
(b) an X-ray powder diffraction (XRPD) pattern with characteristic peaks at 4.4° 2-Theta, 13.0° 2-Theta, 16.0° 2-Theta, 17.0° 2-Theta, 17.7° 2-Theta, 18.7° 2-Theta, 19.3° 2-Theta, 20.9° 2-Theta, 21.7° 2-Theta, and 22.1° 2-Theta;
(c) a thermo-gravimetric analysis (TGA) substantially similar to the one set forth in FIG. 2;
(d) a DSC thermogram substantially similar to the one set forth in FIG. 3;
(e) a DSC thermogram with an endotherm having an onset at about 178° C.;
(f) non-hygroscopicity; or
(g) combinations thereof.

In some embodiments, the crystalline form has an X-ray powder diffraction (XRPD) pattern substantially the same as shown in FIG. 1. In some embodiments, the crystalline form has an X-ray powder diffraction (XRPD) pattern with characteristic peaks at 4.4° 2-Theta, 13.0° 2-Theta, 16.0° 2-Theta, 17.0° 2-Theta, 17.7° 2-Theta, 18.7° 2-Theta, 19.3° 2-Theta, 20.9° 2-Theta, 21.7° 2-Theta, and 22.1° 2-Theta. In some embodiments, the crystalline form has a thermo-gravimetric analysis (TGA) substantially similar to the one set forth in FIG. 2. In some embodiments, the crystalline form has a DSC thermogram substantially similar to the one set forth in FIG. 3. In some embodiments, the crystalline form has a DSC thermogram with an endotherm having an onset at about 178° C. In some embodiments, the crystalline form is non-hygroscopic. In some embodiments, the crystalline form is characterized as having properties (a), (b), (c), (d), (e), and (f). In some embodiments, the crystalline form is obtained from acetone, acetonitrile, anisole, methyl t-butyl ether, dimethoxyethane, 1,4-dioxane, ethanol, ethyl acetate, isopropyl acetate, methanol, methanol/water, ethanol/water, nitromethane, methyl isobutyl ketone, 2-propanol, 2-propanol/water, tetrahydrofuran, tetrahydrofuran/water, tetrahydrofuran/methyl t-butyl ether, toluene, water, heptane, or cumene, or combinations thereof. In some embodiments, the crystalline form is obtained from ethanol. In some embodiments, the crystalline form is obtained from tetrahydrofuran/methyl t-butyl ether. In some embodiments, the crystalline form is unsolvated. In some embodiments, the crystalline form is anhydrous.

In further embodiments are provided pharmaceutical compositions, which include crystalline trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof, and at least one inactive ingredient selected from pharmaceutically acceptable carriers, diluents, and excipients. In some embodiments, the pharmaceutical composition comprises crystalline trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide free base.

In another embodiment, provided herein is a compound that is trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof, for use in medicine.

In another aspect, provided herein is a method of treating or preventing a liver disease or condition in a mammal, comprising administering to the mammal in need thereof a therapeutically effective amount of a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide as described herein. In some embodiments, the disease or condition is a metabolic condition. In some embodiments, the disease or condition is a liver condition.

In some embodiments, the crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide described herein is administered to the mammal by intravenous administration, subcutaneous administration, oral administration, inhalation, nasal administration, dermal administration, or ophthalmic administration.

In another aspect, described herein is a method of treating or preventing any one of the diseases or conditions described herein comprising administering a therapeutically effective amount of a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide described herein, or a pharmaceutically acceptable salt, or solvate thereof, to a mammal in need thereof.

In another aspect, described herein is a method for the treatment or prevention of a metabolic or liver condition in a mammal comprising administering a therapeutically effective amount of a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide described herein, or a pharmaceutically acceptable salt, or solvate thereof, to the mammal in need thereof. In other embodiments, the metabolic or liver condition is amenable to treatment with a FXR agonist. In some embodiments, the method further comprises administering a second therapeutic agent to the mammal in addition to the crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide described herein, or a pharmaceutically acceptable salt, or solvate thereof.

In another aspect, described herein is a method of treating or preventing a liver disease or condition in a mammal, comprising administering to the mammal a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof. In some embodiments, the liver disease or condition is an alcoholic or non-alcoholic liver disease. In some embodiments, the liver disease or condition is primary biliary cirrhosis, primary sclerosing cholangitis, cholestasis, nonalcoholic steatohepatitis (NASH), or nonalcoholic fatty liver disease (NAFLD). In some embodiments, the alcoholic liver disease or condition is fatty liver (steatosis), cirrhosis, or alcoholic hepatitis. In some embodiments, the non-alcoholic liver disease or condition is nonalcoholic steatohepatitis (NASH), or nonalcoholic fatty liver disease (NAFLD). In some embodiments, the non-alcoholic liver disease or condition is nonalcoholic steatohepatitis (NASH). In some embodiments, the non-alcoholic liver disease or condition is nonalcoholic steatohepatitis (NASH) and is accompanied by liver fibrosis. In some embodiments, the non-alcoholic liver disease or condition is nonalcoholic steatohepatitis (NASH) without liver fibrosis. In some embodiments, the non-alcoholic liver disease or condition is intrahepatic cholestasis or extrahepatic cholestasis.

In another aspect, described herein is a method of treating or preventing a liver fibrosis in a mammal, comprising administering to the mammal a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof. In some embodiments, the mammal is diagnosed with hepatitis C virus (HCV), nonalcoholic steatohepatitis (NASH), primary sclerosing cholangitis (PSC), cirrhosis, Wilson's disease, hepatitis B virus (HBV), HIV associated steatohepatitis and cirrhosis, chronic viral hepatitis, non-alcoholic fatty liver disease (NAFLD), alcoholic steatohepatitis (ASH), nonalcoholic steatohepatitis (NASH), primary biliary cirrhosis (PBC), or biliary cirrhosis. In some embodiments, the mammal is diagnosed with nonalcoholic steatohepatitis (NASH).

In another aspect, described herein is a method of treating or preventing a liver inflammation in a mammal, comprising administering to the mammal a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof. In some embodiments, the mammal is diagnosed with hepatitis C virus (HCV), nonalcoholic steatohepatitis (NASH), primary sclerosing cholangitis (PSC), cirrhosis, Wilson's disease, hepatitis B virus (HBV), HIV associated steatohepatitis and cirrhosis, chronic viral hepatitis, non-alcoholic fatty liver disease (NAFLD), alcoholic steatohepatitis (ASH), nonalcoholic steatohepatitis (NASH), primary biliary cirrhosis (PBC), or biliary cirrhosis. In some embodiments, the mammal is diagnosed with nonalcoholic steatohepatitis (NASH). In some embodiments, the liver inflammation is associated with inflammation in the gastrointestinal tract. In some embodiments, the mammal is diagnosed with inflammatory bowel disease.

In another aspect, described herein is a method of treating or preventing a gastrointestinal disease or condition in a mammal, comprising administering to the mammal a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof. In some embodiments, the gastrointestinal disease or condition is necrotizing enterocolitis, gastritis, ulcerative colitis, Crohn's disease, inflammatory bowel disease, irritable bowel syndrome, gastroenteritis, radiation induced enteritis, pseudomembranous colitis, chemotherapy induced enteritis, gastro-esophageal reflux disease (GERD), peptic ulcer, non-ulcer dyspepsia (NUD), celiac disease, intestinal celiac disease, post-surgical inflammation, gastric carcinogenesis, graft versus host disease or any combination thereof. In some embodiments, the gastrointestinal disease is irritable bowel syndrome (IBS), irritable bowel syndrome with diarrhea (IBS-D), irritable bowel syndrome with constipation (IBS-C), mixed IBS (IBS-M), unsubtyped IBS (IBS-U), or bile acid diarrhea (BAD)

In another aspect, described herein is a method of treating or preventing a disease or condition in a mammal that would benefit from treatment with a FXR agonist, comprising administering to the mammal a crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof. In some embodiments, the methods described herein further comprise administering at least one additional therapeutic agent in addition to the crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof.

In another aspect, provided herein is a spray-dried solid dispersion comprising: (a) trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, and (b) a pharmaceutically acceptable polymer; wherein trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide is dispersed in a polymer matrix formed from the pharmaceutically acceptable polymer. In some embodiments, the pharmaceutically acceptable polymer is selected from PVP/VA 64, PVP 30, HPMC-AS M, HPMCAS-L, Eudragit L100-55, Eudragit L100, Eudragit EPO, HPMC E15, HPMC E3, HPMCP-HP55, PVA and Soluplus. In some embodiments, the pharmaceutically acceptable polymer is selected from PVP/VA 64, PVP 30, HPMC-AS M, Eudragit L100-55, Eudragit L100, and HPMC E15. In some embodiments, the pharmaceutically acceptable polymer is Eudragit L100. In some embodiments, the pharmaceutically acceptable polymer is PVP/VA 64. In some embodiments, the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is from 9:1 to 1:9. In some embodiments, the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is from 4:1 to 1:3. In some embodiments, the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is 4:1. In some embodiments, the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is 1:1. In some embodiments, the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is 3:7. In some embodiments, the spray-dried solid dispersion further comprises a non-aqueous solvent. In some embodiments, the non-aqueous solvent is selected from the group consisting of tert-butanol, n-propanol, n-butanol, isopropanol, ethanol, methanol, acetone, ethyl acetate, dimethyl carbonate, acetonitrile, dichloromethane, methyl ethyl ketone, methyl isobutyl ketone, 1-pentanol, methyl acetate, carbon tetrachloride, dimethyl sulfoxide, hexafluoroacetone, chlorobutanol, dimethyl sulfone, acetic acid, cyclohexane, and mixtures thereof. In some embodiments, the non-aqueous solvent is selected from the group consisting of ethanol, methanol, propanol, butanol, isopropanol, tert-butanol, dichloromethane, and mixtures thereof. In some embodiments, the non-aqueous solvent is a mixture of dichloromethane and methanol. In some embodiments of the spray-dried solid dispersion, trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide is substantially amorphous.

In another aspect, provided herein is a pharmaceutical formulation comprising a spray-dried solid dispersion described herein, further comprising one or more pharmaceutical acceptable ingredients selected from the group consisting of one or more diluents, one or more disintegrants, one or more binders, one or more lubricants, one or more glidants, and one or more surfactants. In some embodiments, the one or more pharmaceutical acceptable ingredients are selected from the group consisting of microcrystalline cellulose, lactose monohydrate, croscarmellose sodium, magnesium stearate, colloidal silicon dioxide, mannitol, crospovidone, and sodium stearyl fumarate. In some embodiments, the one or more pharmaceutical acceptable ingredients are selected from the group consisting of microcrystalline cellulose, lactose monohydrate, croscarmellose sodium, magnesium stearate, and colloidal silicon dioxide. In some embodiments, the pharmaceutical formulation is in tablet form. In some embodiments, the pharmaceutical formulation is in capsule form.

In another aspect, provided herein is a method of treating or preventing a liver disease or condition in a mammal, comprising administering to the mammal in need thereof a therapeutically effective amount of a spray-dried solid dispersion as described herein. In some embodiments, the disease or condition is a metabolic condition. In some embodiments, the disease or condition is a liver condition.

In some embodiments, the spray-dried solid dispersion described herein is administered to the mammal by intravenous administration, subcutaneous administration, oral administration, inhalation, nasal administration, dermal administration, or ophthalmic administration.

In another aspect, described herein is a method of treating or preventing any one of the diseases or conditions described herein comprising administering a therapeutically effective amount of a spray-dried solid dispersion described herein, or a pharmaceutically acceptable salt, or solvate thereof, to a mammal in need thereof.

In another aspect, described herein is a method for the treatment or prevention of a metabolic or liver condition in a mammal comprising administering a therapeutically effective amount of a spray-dried solid dispersion described herein, or a pharmaceutically acceptable salt, or solvate thereof, to the mammal in need thereof. In other embodiments, the metabolic or liver condition is amenable to treatment with a FXR agonist. In some embodiments, the method further comprises administering a second therapeutic agent to the mammal in addition to the spray-dried solid dispersion described herein, or a pharmaceutically acceptable salt, or solvate thereof.

In another aspect, described herein is a method of treating or preventing a liver disease or condition in a mammal, comprising administering to the mammal a spray-dried solid dispersion described herein. In some embodiments, the liver disease or condition is an alcoholic or non-alcoholic liver disease. In some embodiments, the liver disease or condition is primary biliary cirrhosis, primary sclerosing cholangitis, cholestasis, nonalcoholic steatohepatitis (NASH), or nonalcoholic fatty liver disease (NAFLD). In some embodiments, the alcoholic liver disease or condition is fatty liver (steatosis), cirrhosis, or alcoholic hepatitis. In some embodiments, the non-alcoholic liver disease or condition is nonalcoholic steatohepatitis (NASH), or nonalcoholic fatty liver disease (NAFLD). In some embodiments, the non-alcoholic liver disease or condition is nonalcoholic steatohepatitis (NASH). In some embodiments, the non-alcoholic liver disease or condition is nonalcoholic steatohepatitis (NASH) and is accompanied by liver fibrosis. In some embodiments, the non-alcoholic liver disease or condition is nonalcoholic steatohepatitis (NASH) without liver fibrosis. In some embodiments, the non-alcoholic liver disease or condition is intrahepatic cholestasis or extrahepatic cholestasis.

In another aspect, described herein is a method of treating or preventing a liver fibrosis in a mammal, comprising administering to the mammal a spray-dried solid dispersion described herein. In some embodiments, the mammal is diagnosed with hepatitis C virus (HCV), nonalcoholic steatohepatitis (NASH), primary sclerosing cholangitis (PSC), cirrhosis, Wilson's disease, hepatitis B virus (HBV), HIV associated steatohepatitis and cirrhosis, chronic viral hepatitis, non-alcoholic fatty liver disease (NAFLD), alcoholic steatohepatitis (ASH), nonalcoholic steatohepatitis (NASH), primary biliary cirrhosis (PBC), or biliary cirrhosis. In some embodiments, the mammal is diagnosed with nonalcoholic steatohepatitis (NASH).

In another aspect, described herein is a method of treating or preventing a liver inflammation in a mammal, comprising administering to the mammal a spray-dried solid dispersion described herein. In some embodiments, the mammal is diagnosed with hepatitis C virus (HCV), nonalcoholic steatohepatitis (NASH), primary sclerosing cholangitis (PSC), cirrhosis, Wilson's disease, hepatitis B virus (HBV), HIV associated steatohepatitis and cirrhosis, chronic viral hepatitis, non-alcoholic fatty liver disease (NAFLD), alcoholic steatohepatitis (ASH), nonalcoholic steatohepatitis (NASH), primary biliary cirrhosis (PBC), or biliary cirrhosis. In some embodiments, the mammal is diagnosed with nonalcoholic steatohepatitis (NASH). In some embodiments, the liver inflammation is associated with inflammation in the gastrointestinal tract. In some embodiments, the mammal is diagnosed with inflammatory bowel disease.

In another aspect, described herein is a method of treating or preventing a gastrointestinal disease or condition in a mammal, comprising administering to the mammal a spray-dried solid dispersion described herein. In some embodiments, the gastrointestinal disease or condition is necrotizing enterocolitis, gastritis, ulcerative colitis, Crohn's disease, inflammatory bowel disease, irritable bowel syndrome, gastroenteritis, radiation induced enteritis, pseudomembranous colitis, chemotherapy induced enteritis, gastro-esophageal reflux disease (GERD), peptic ulcer, non-ulcer dyspepsia (NUD), celiac disease, intestinal celiac disease, post-surgical inflammation, gastric carcinogenesis, graft versus host disease or any combination thereof. In some embodiments, the gastrointestinal disease is irritable bowel syndrome (IBS), irritable bowel syndrome with diarrhea (IBS-D), irritable bowel syndrome with constipation (IBS-C), mixed IBS (IBS-M), unsubtyped IBS (IBS-U), or bile acid diarrhea (BAD)

In another aspect, described herein is a method of treating or preventing a disease or condition in a mammal that would benefit from treatment with a FXR agonist, comprising administering to the mammal a spray-dried solid dispersion described herein. In some embodiments, the methods described herein further comprise administering at least one additional therapeutic agent in addition to the spray-dried solid dispersion described herein.

### INCORPORATION BY REFERENCE

All publications and patent applications mentioned in this specification are herein incorporated by reference to the extent applicable and relevant.

### BRIEF DESCRIPTION OF THE FIGURES

FIG. 1 illustrates an X-ray powder diffraction (XRPD) pattern of Form 1 of crystalline trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide free base.

FIG. 2 illustrates a thermo-gravimetric analysis (TGA) thermogram of Form 1 of crystalline trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide free base.

FIG. 3 illustrates a differential scanning calorimetry (DSC) thermogram of Form 1 of crystalline trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide free base (upper trace at right-hand margin of graph).

FIG. 4 illustrates a gravimetric vapor sorption (GVS) isotherm (dynamic vapor sorption (DVS) isotherm plot) over two complete sorption/desorption cycles of Form 1 of crystalline trans-N-(3-(1-cyclopropyl-1H-pyrazol-4-yl)phenyl)-4-hydroxy-N-((trans-4-(4-methoxy-3-methylphenyl)-cyclohexyl)methyl)cyclohexanecarboxamide free base.

FIG. 5 illustrates a gravimetric vapor sorption (GVS) kinetic plot (dynamic vapor sorption (DVS) mass plot) over two complete sorption/desorption cycles of Form 1 of crystalline trans-N-(3-(1-cyclopropyl-1H-pyrazol-4-yl)phenyl)-4-hydroxy-N-((trans-4-(4-methoxy-3-methylphenyl)-cyclohexyl)methyl)cyclohexanecarboxamide free base.

FIG. 6 Illustrates X-ray powder diffraction (XRPD) patterns of Form 1 of crystalline trans-N-(3-(1-cyclopropyl-1H-pyrazol-4-yl)phenyl)-4-hydroxy-N-((trans-4-(4-methoxy-3-methylphenyl)-cyclohexyl)methyl)cyclohexanecarboxamide free base (bottom trace to top trace): a) as prepared by maturation in ethanol; b) after 7 days at 25° C. and 97% RH; c) after 7 days at 40° C. and 75% RH; and d) after GVS.

FIG. 7 illustrates HPLC chromatograms of Form 1 of crystalline trans-N-(3-(1-cyclopropyl-1H-pyrazol-4-yl)phenyl)-4-hydroxy-N-((trans-4-(4-methoxy-3-methylphenyl)cyclohexyl)methyl)-cyclohexanecarboxamide free base: a) as prepared by maturation in ethanol (upper left); b) after 7 days at 25° C. and 97% RH (lower right); and c) after 7 days at 40° C. and 75% RH (upper right).

FIG. 8 illustrates X-ray powder diffraction (XRPD) patterns of Form 1B of crystalline trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide free base: a) vacuum-dried at room temperature overnight sample (top); and b) wet sample (bottom).

FIG. 9 illustrates an X-ray powder diffraction (XRPD) patterns of Form 1C of crystalline trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide free base: a) vacuum-dried at 25° C. overnight sample (top); and b) wet sample (bottom).

FIG. 10 illustrates the molecular configuration of Form 1 of Compound 1. Anisotropic atomic displacement ellipsoids for the non-hydrogen atoms are shown at the 50% probability level.

### DETAILED DESCRIPTION OF THE INVENTION

The nuclear hormone receptor farnesoid X receptor (also known as FXR or nuclear receptor subfamily 1, group H, member 4 (NR1H4)) (OMIM: 603826) functions as a regulator for bile acid metabolism. FXR is a ligand-activated transcriptional receptor expressed in diverse tissues including the adrenal gland, kidney, stomach, duodenum, jejunum, ileum, colon, gall bladder, liver, macrophages, and white and brown adipose tissue. FXRs are highly expressed in tissues that participate in bile acid metabolism such as the liver, intestines, and kidneys. Bile acids function as endogenous ligands for FXR such that enteric and systemic release of bile acids induces FXR-directed changes in gene expression networks. Bile acids are the primary oxidation product of cholesterol, and in some cases, upon secretion into the intestines, are regulators of cholesterol absorption. The rate-limiting step for conversion of cholesterol into bile acids is catalyzed by cytochrome p450 enzyme cholesterol 7-α-hydroxylase (CYP7A1) and occurs in the liver. The cytochrome p450 enzyme sterol 12-α-hydroxylase (CYP8B1) mediates production of cholic acid and determines the relative amounts of the two primary bile acids, cholic acid and chenodeoxycholic acid. Activation of FXR can represses the transcription of CYP7A1 and CYP8B1 by increasing the expression level of the hepatic small heterodimer partner (SHP) (also known as nuclear receptor subfamily 0, group B, member 2; or NR0B2) and intestinal expression of fibroblast growth factor 15 (FGF15) in mice and fibroblast growth factor 19 (FGF19) in human. SHP represses the liver receptor homolog (LRH-1) and hepatocyte nuclear factor 4alpha (HNFa4), transcription factors that regulate CYP7A1 and CYP8B1 gene expression. CYP8B1 repression by FXR can be species-specific and FXR activation may in some cases increase CYP8B1 expression in humans (Sanyal et al PNAS, 2007, 104, 15665). In some cases, FGF15/19 released from the intestine then activates the fibroblast growth factor receptor 4 in the liver, leading to activation of the mitogen-activated protein kinase (MAPK) signaling pathway which suppress CYP7A1 and CYP8B1.

In some embodiments, elevated levels of bile acids have been associated with insulin resistance. For example, insulin resistance sometimes leads to a decreased uptake of glucose from the blood and increased de novo glucose production in the liver. In some instances, intestinal sequestration of bile acids has been shown to improve insulin resistance by promoting the secretion of glucagon-like peptide-1 (GLP1) from intestinal L-cells. GLP-1 is an incretin derived from the transcription product of the proglucagon gene. It is released in response to the intake of food and exerts control in appetite and gastrointestinal function and promotes insulin secretion from the pancreas. The biologically active forms of GLP-1 include GLP-1-(7-37) and GLP-1-(7-36)NH₂, which result from selective cleavage of the proglucagon molecule. In such cases, activation of FXR leading to decreased production of bile acids correlates to a decrease in insulin resistance.

In some embodiments, the activation of FXR also correlates to the secretion of pancreatic polypeptide-fold such as peptide YY (PYY or PYY3-36). In some instances, peptide YY is a gut hormone peptide that modulates neuronal activity within the hypothalamic and brainstem, regions of the brain involved in reward processing. In some instances, reduced level of PYY correlates to increased appetite and weight gain.

In some instances, the activation of FXR indirectly leads to a reduction of plasma triglycerides. The clearance of triglycerides from the bloodstream is due to lipoprotein lipase (LPL). LPL activity is enhanced by the induction of its activator apolipoprotein CII, and the repression of its inhibitor apolipoprotein CIII in the liver occurs upon FXR activation.

In some cases, the activation of FXR further modulates energy expenditure such as adipocyte differentiation and function. Adipose tissue comprises adipocytes or fat cells. In some instances, adipocytes are further differentiated into brown adipose tissue (BAT) or white adipose tissue (WAT). The function of BAT is to generate body heat, while WAT functions as fat storing tissues.

In some instances, FXR is widely expressed in the intestine. In some cases, the activation of FXR has been shown to induce the expression and secretion of FGF19 (or FGF15 in mouse) in the intestine. FGF19 is a hormone that regulates bile acid synthesis as well as exerts an effect on glucose metabolism, lipid metabolism, and on energy expenditure. In some instances, FGF19 has also been observed to modulate adipocyte function and differentiation. Indeed, a study has shown that the administration of FGF19 to high-fat diet-fed mice increased energy expenditure, modulated adipocytes differentiation and function, reversed weight gain, and improved insulin resistance (see, Fu et al., “Fibroblast growth factor 19 increases metabolic rate and reverses dietary and leptin-deficient diabetes.” Endocrinology 145:2594-2603 (2004)).

In some cases, intestinal FXR activity has also been shown to be involved in reducing overgrowth of the microbiome, such as during feeding (Li et al., Nat Commun 4:2384, 2013). For example, a study had shown that activation of FXR correlated with increased expression of several genes in the ileum such as Ang2, iNos, and 1118, which have established antimicrobial actions (Inagaki et al., Proc Natl Acad Sci USA 103:3920-3925, 2006).

In some cases, FXR has been implicated in barrier function and immune modulation in the intestine. FXR modulates transcription of genes involved in bile salt synthesis, transport and metabolism in the liver and intestine, and in some cases has been shown to lead to improvements in intestinal inflammation and prevention of bacterial translocation into the intestinal tract (Gadaleta et al., Gut. 2011 April; 60(4):463-72).

In some cases, over production of bile acids or improper transport and re-cycling of bile acids can lead to diarrhea. FXR modulates transcription of genes involved in bile salt synthesis, transport and metabolism in the liver and intestine, and in some cases may lead to improvements in diarrhea Camilleri, Gut Liver. 2015 May; 9(3): 332-339.

G protein-coupled bile acid receptor 1 (also known as GPBAR2, GPCR19, membrane-type receptor for bile acids or M-BAR, or TGR5) is a cell surface receptor for bile acids. Upon activation with bile acid, TGR5 induces the production of intracellular cAMP, which then triggers an increase in triiodothyronine due to the activation of deiodinase (DIO2) in BAT, resulting in increased energy expenditure.

Hence in some embodiments, regulation of metabolic processes such as bile acid synthesis, bile-acid circulation, glucose metabolism, lipid metabolism, or insulin sensitivity is modulated by the activation of FXR. Furthermore, in some embodiments, dis-regulation of metabolic processes such as bile acid synthesis, bile-acid circulation, glucose metabolism, lipid metabolism, or insulin sensitivity results in metabolic diseases such as diabetes or diabetes-related conditions or disorders, alcoholic or non-alcoholic liver disease or condition, intestinal inflammation, or cell proliferative disorders.

Disclosed herein, in certain embodiments, are compounds that have activity as FXR agonists. In some embodiments, the FXR agonists described herein are structurally distinct from bile acids, other synthetic FXR ligands, and other natural FXR ligands.

In some embodiments, also disclosed herein are methods of treating or preventing a metabolic disorder, such as diabetes, obesity, impaired glucose tolerance, dyslipidemia, or insulin resistance by administering a therapeutically effective amount of an FXR agonist. In some instances, the compounds are administered to the GI tract of a subject.

In additional embodiments, disclosed herein are methods for treating or preventing alcoholic or non-alcoholic liver disease or conditions (e.g., cholestasis, primary biliary cirrhosis, steatosis, cirrhosis, alcoholic hepatitis, non-alcoholic steatohepatitis (NASH), non-alcoholic fatty liver disease (NAFLD), primary sclerosing cholangitis (PSC) or elevated liver enzymes) by administering a therapeutically effective amount of an FXR agonist to a subject in need thereof (e.g., via the GI tract). In additional embodiments, disclosed herein include methods for treating or preventing cholestasis, cirrhosis, primary biliary cirrhosis, non-alcoholic steatohepatitis (NASH), non-alcoholic fatty liver disease (NAFLD), or primary sclerosing cholangitis (PSC) by administering a therapeutically effective amount of an FXR agonist to a subject in need thereof. In some embodiments, disclosed herein include methods for treating or preventing cholestasis by administering a therapeutically effective amount of an FXR agonist to a subject in need thereof. In some embodiments, disclosed herein include methods for treating or preventing primary biliary cirrhosis by administering a therapeutically effective amount of an FXR agonist to a subject in need thereof. In some embodiments, disclosed herein include methods for treating or preventing NASH by administering a therapeutically effective amount of an FXR agonist to a subject in need thereof. In some embodiments, disclosed herein include methods for treating or preventing NAFLD by administering a therapeutically effective amount of an FXR agonist to a subject in need thereof.

In further embodiments, disclosed herein include methods for treating or preventing inflammation in the intestines and/or a cell proliferative disorder, such as cancer, by administering a therapeutically effective amount of an FXR agonist to a subject in need thereof (e.g., via the GI tract).

In still further embodiments, disclosed herein include FXR agonists that modulate one or more of the proteins or genes associated with a metabolic process such as bile acid synthesis, glucose metabolism, lipid metabolism, or insulin sensitivity, such as for example, increase in the activity of FGF19 (FGF15 in mouse), increase in the secretion of GLP-1, or increase in the secretion of PYY.

### trans-N-(3-(1-Cyclopropyl-1H-pyrazol-4-yl)phenyl)-4-hydroxy-N-((trans-4-(4-methoxy-3-methylphenyl)cyclohexyl)methyl)cyclohexanecarboxamide (Compound 1)

Described herein is the FXR agonist compound, trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide (Compound 1). “Compound 1” or “trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide” refers to the compound with the following structure:

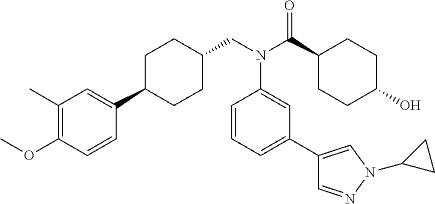 [SMILES: COc1ccc([C@H]2CC[C@H](CN(c3cccc(-c4cnn(C5CC5)c4)c3)C(=O)[C@H]3CC[C@H](O)CC3)CC2)cc1C]

In some embodiments, Compound 1 is in the form of pharmaceutically acceptable salt. In some embodiments, Compound 1 is a free base. In addition, Compound 1 can exist in unsolvated as well as solvated forms with pharmaceutically acceptable solvents such as water, ethanol, and the like. The solvated forms of Compound 1 presented herein are also considered to be disclosed herein. In some embodiments, Compound 1 is solvated. In some embodiments, Compound 1 is unsolvated.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

### Amorphous Compound 1

In some embodiments, Compound 1 is amorphous. In some embodiments, Compound 1 is amorphous and anhydrous. In some embodiments, amorphous Compound 1 has an X-ray powder diffraction (XRPD) pattern showing a lack of crystallinity.

### Crystalline Forms of Compound 1

The identification and selection of a solid form of a pharmaceutical compound are complex, given that a change in solid form may affect a variety of physical and chemical properties, which may provide benefits or drawbacks in processing, formulation, stability, bioavailability, storage, handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable and marketable pharmaceutical product.

### Crystalline Form 1 of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide (Compound 1)

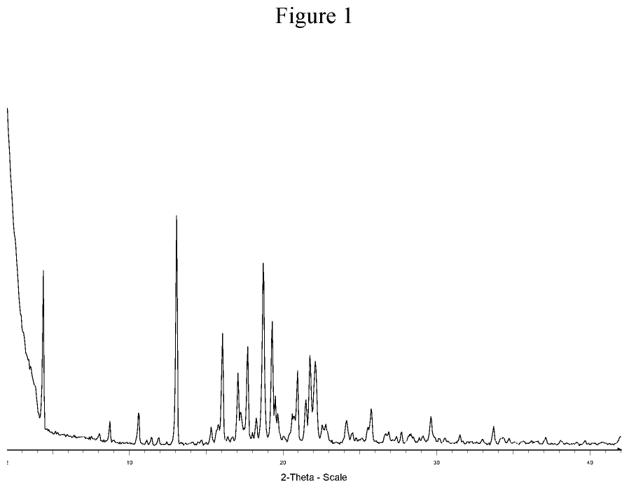

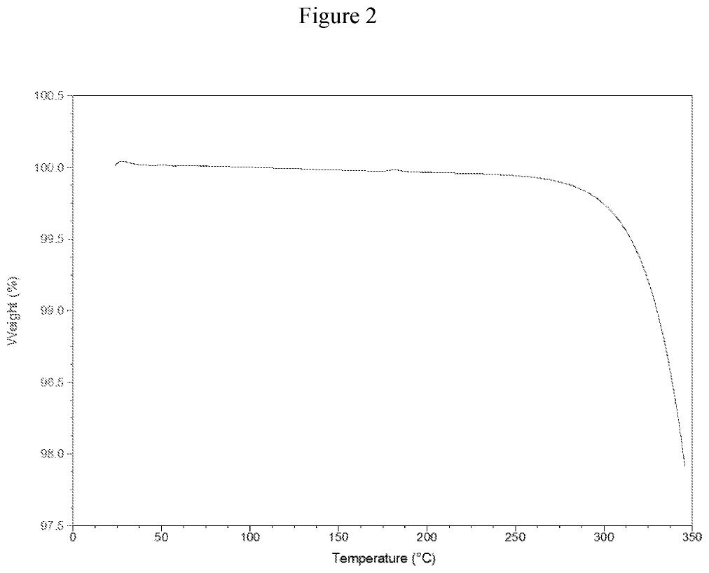

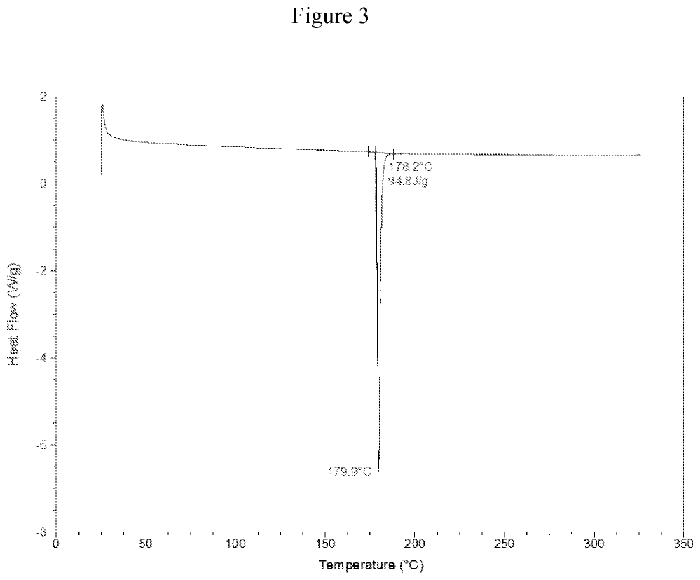

In some embodiments, trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide (Compound 1) is crystalline. In some embodiments, crystalline Compound 1 is characterized as having at least one of the following properties:
(a) an X-ray powder diffraction (XRPD) pattern substantially the same as shown in FIG. 1;
(b) an X-ray powder diffraction (XRPD) pattern with characteristic peaks at 4.4° 2-Theta, 13.0° 2-Theta, 16.0° 2-Theta, 17.0° 2-Theta, 17.7° 2-Theta, 18.7° 2-Theta, 19.3° 2-Theta, 20.9° 2-Theta, 21.7° 2-Theta, and 22.1° 2-Theta;
(c) a thermo-gravimetric analysis (TGA) substantially similar to the one set forth in FIG. 2;
(d) a DSC thermogram substantially similar to the one set forth in FIG. 3;
(e) a DSC thermogram with an endotherm having an onset at about 178° C.;
(f) non-hygroscopicity; or
(g) combinations thereof.

In some embodiments, crystalline Compound 1 is characterized as having at least two of the properties selected from (a) to (f). In some embodiments, crystalline Compound 1 is characterized as having at least three of the properties selected from (a) to (f). In some embodiments, crystalline Compound 1 is characterized as having at least four of the properties selected from (a) to (f). In some embodiments, crystalline Compound 1 is characterized as having at least five of the properties selected from (a) to (f). In some embodiments, crystalline Compound 1 is characterized as having properties (a) to (f).

In some embodiments, crystalline Compound 1 has an X-ray powder diffraction (XRPD) pattern substantially the same as shown in FIG. 1. In some embodiments, crystalline Compound 1 has an X-ray powder diffraction (XRPD) pattern with characteristic peaks at 4.4° 2-Theta, 13.0° 2-Theta, 16.0° 2-Theta, 17.0° 2-Theta, 17.7° 2-Theta, 18.7° 2-Theta, 19.3° 2-Theta, 20.9° 2-Theta, 21.7° 2-Theta, and 22.1° 2-Theta. In some embodiments, crystalline Compound 1 has a thermo-gravimetric analysis (TGA) thermogram substantially similar to the one set forth in FIG. 2. In some embodiments, crystalline Compound 1 has a DSC thermogram substantially similar to the one set forth in FIG. 3. In some embodiments, crystalline Compound 1 has a DSC thermogram with an endotherm having an onset at about 178° C. In some embodiments, the crystalline Compound 1 is non-hygroscopic. In some embodiments, crystalline Compound 1 is obtained from acetone, acetonitrile, anisole, methyl t-butyl ether, dimethoxyethane, 1,4-dioxane, ethanol, ethyl acetate, isopropyl acetate, methanol, methanol/water, ethanol/water, nitromethane, methyl isobutyl ketone, 2-propanol, 2-propanol/water, tetrahydrofuran, tetrahydrofuran/water, tetrahydrofuran/methyl t-butyl ether, toluene, water, heptane, or cumene, or combinations thereof. In some embodiments, crystalline Compound 1 is obtained from ethanol. In some embodiments, crystalline Compound 1 is obtained from tetrahydrofuran/methyl t-butyl ether. In some embodiments, the crystalline Compound 1 is solvated. In some embodiments, crystalline Compound 1 is unsolvated. In some embodiments, crystalline Compound 1 is hydrated. In some embodiments, crystalline Compound 1 is anhydrous.

### Preparation of Crystalline Forms

In some embodiments, crystalline forms of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide (Compound 1) are prepared as outlined in the Examples. It is noted that solvents, temperatures and other reaction conditions presented herein may vary.

In certain embodiments, provided herein are methods for making a crystalline form of Compound 1, comprising 1) suspending Compound 1 in a solvent at a first temperature (e.g., room temperature); 2) placing the resulting mixture in a maturation chamber to cycle between room temperature and a second temperature (e.g., 50° C.) for a certain time and certain frequency (e.g., 5 days, 4 hours at each temperature); 3) collecting a solid if a solid is present at the certain time; and 4) optionally drying the collected solid. In certain embodiments, provided herein are methods for making a solid form of Compound 1, comprising 1) obtaining a saturated solution of Compound 1 in a solvent at about 60° C.; 2) adding an anti-solvent into the saturated solution at about 60° C.; 3) cooling down to about 5° C.; and 4) collecting a solid, optionally by suction filtration; and 5) optionally vacuum drying.

In another embodiment, crystalline Compound 1 is substantially pure. In certain embodiments, the substantially pure crystalline Compound 1 is substantially free of other solid forms, e.g., amorphous solid. In certain embodiments, the purity of the substantially pure crystalline Compound 1 is no less than about 95%, no less than about 96%, no less than about 97%, no less than about 98%, no less than about 98.5%, no less than about 99%, no less than about 99.5%, or no less than about 99.8%.

### Compound 1 Co-Crystals

Co-crystals are crystalline molecular complexes of two or more non-volatile compounds bound together in a crystal lattice by non-ionic interactions. Pharmaceutical co-crystals are co-crystals of a therapeutic compound, e.g., Compound 1, and one or more non-volatile compound(s). The one or more non-volatile compound in a pharmaceutical cocrystal is typically selected from nontoxic pharmaceutically acceptable molecules, such as, for example, food additives, preservatives, pharmaceutical excipients, or other APIs. In some embodiments, provided herein is a co-crystal comprising Compound 1, or a pharmaceutically acceptable salt or solvate thereof, and at least one inactive ingredient selected from pharmaceutically acceptable carriers, diluents, and excipients. In some embodiments, co-crystals are prepared using solid-state methods such as solid-state grinding and solvent-drop grinding. In some embodiments, co-crystals are prepared using high-throughput screening. In some embodiments, co-crystals are prepared using solution-based crystallization. In some embodiments, co-crystals formation leads to enhancement of physical properties of the resulting solid forms, such as solubility, dissolution rate, bioavailablity, physical stability, chemical stability, flowability, fractability, or compressibility. In some embodiments, Compound 1 forms different co-crystals with different counter-molecules, and some of these co-crystals exhibit enhanced solubility or stability. In some embodiments pharmaceutical co-crystals of Compound 1 increase the bioavailability or stability profile of Compound 1.

### Suitable Solvents

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, methyl t-butyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound 1 comprise an organic solvent(s). In some embodiments, compositions comprising Compound 1 comprise a residual amount of an organic solvent(s). In some embodiments, the organic solvent is a Class 3 solvent. In some embodiments, compositions comprising Compound 1 comprise a residual amount of a Class 3 solvent. In some embodiments, the Class 3 solvent is selected from the group consisting of acetic acid, acetone, anisole, 1-butanol, 2-butanol, butyl acetate, methyl t-butyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from ethyl acetate, isopropyl acetate, tert-butylmethylether, heptane, isopropanol, and ethanol.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. Compound 1, or a pharmaceutically acceptable salt thereof, and a co-agent, are both administered to a patient simultaneously in the form of a single entity or dosage. The term “non-fixed combination” means that the active ingredients, e.g. Compound 1, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, Compound 1 described herein is formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, Compound 1 described herein is administered either alone or in combination with pharmaceutically acceptable carriers, excipients or diluents, in a pharmaceutical composition. Administration of Compound 1 described herein, and pharmaceutical compositions thereof, can be affected by any method that enables delivery of the compound to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, Compound 1 can be administered locally to the area in need of treatment, by for example, local infusion during surgery, topical application such as creams or ointments, injection, catheter, or implant. The administration can also be by direct injection at the site of a diseased tissue or organ.

In some embodiments, Compound 1 pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

It should be understood that in addition to the ingredients particularly mentioned above, the compositions described herein may include other agents conventional in the art having regard to the type of formulation in question, for example those suitable for oral administration may include flavoring agents.

### Compound 1 Spray Dried Dispersion Formulations

In some embodiments described herein, the Compound 1 pharmaceutical composition is a spray dried dispersion formulation. In some embodiments, provided herein is a spray-dried solid dispersion comprising: (a) trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, and (b) a pharmaceutically acceptable polymer; wherein trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide is dispersed in a polymer matrix formed from the pharmaceutically acceptable polymer. In some embodiments, the pharmaceutically acceptable polymer is selected from PVP/VA 64, PVP 30, HPMC-AS M, HPMCAS-L, Eudragit L100-55, Eudragit L100, Eudragit EPO, HPMC E15, HPMC E3, HPMCP-HP55, PVA and Soluplus. In some embodiments, the pharmaceutically acceptable polymer is selected from PVP/VA 64, PVP 30, HPMC-AS M, Eudragit L100-55, Eudragit L100, and HPMC E15. In some embodiments, the pharmaceutically acceptable polymer is PVP/VA 64. In some embodiments, the pharmaceutically acceptable polymer is PVP 30. In some embodiments, the pharmaceutically acceptable polymer is HPMC-AS M. In some embodiments, the pharmaceutically acceptable polymer is Eudragit L100-55. In some embodiments, the pharmaceutically acceptable polymer is Eudragit L100. In some embodiments, the pharmaceutically acceptable polymer is HPMC E15. In some embodiments, the weight ratio of Compound 1 to the pharmaceutically acceptable polymer is from 9:1 to 1:9. In some embodiments, the weight ratio of Compound 1 to the pharmaceutically acceptable polymer is from 7:1 to 1:7. In some embodiments, the weight ratio of Compound 1 to the pharmaceutically acceptable polymer is from 5:1 to 1:5. In some embodiments, the weight ratio of Compound 1 to the pharmaceutically acceptable polymer is from 4:1 to 1:3. In some embodiments, the weight ratio of Compound 1 to the pharmaceutically acceptable polymer is from 2:1 to 1:2. In some embodiments, the weight ratio of Compound 1 to the pharmaceutically acceptable polymer is 4:1. In some embodiments, the weight ratio of Compound 1 to the pharmaceutically acceptable polymer is 3:1. In some embodiments, the weight ratio of Compound 1 to the pharmaceutically acceptable polymer is 2:1. In some embodiments, the weight ratio of Compound 1 to the pharmaceutically acceptable polymer is 1:1. In some embodiments, the weight ratio of Compound 1 to the pharmaceutically acceptable polymer is 1:2. In some embodiments, the weight ratio of Compound 1 to the pharmaceutically acceptable polymer is 3:7. In some embodiments, the weight ratio of Compound 1 to the pharmaceutically acceptable polymer is 1:3. In some embodiments, the spray-dried solid dispersion further comprises a non-aqueous solvent. In some embodiments, the non-aqueous solvent is selected from the group consisting of tert-butanol, n-propanol, n-butanol, isopropanol, ethanol, methanol, acetone, ethyl acetate, dimethyl carbonate, acetonitrile, dichloromethane, methyl ethyl ketone, methyl isobutyl ketone, 1-pentanol, methyl acetate, carbon tetrachloride, dimethyl sulfoxide, hexafluoroacetone, chlorobutanol, dimethyl sulfone, acetic acid, cyclohexane, and mixtures thereof. In some embodiments, the non-aqueous solvent is selected from the group consisting of ethanol, methanol, propanol, butanol, isopropanol, tert-butanol, dichloromethane, and mixtures thereof. In some embodiments, the non-aqueous solvent is a mixture of dichloromethane and methanol. In some embodiments, the non-aqueous solvent is a mixture of dichloromethane and methanol, wherein the weight ratio of dichloromethane to methanol is 4/1. In some embodiments, the non-aqueous solvent is a mixture of dichloromethane and methanol, wherein the weight ratio of dichloromethane to methanol is 7/3. In some embodiments, the non-aqueous solvent is a mixture of dichloromethane and methanol, wherein the weight ratio of dichloromethane to methanol is 3/2. In some embodiments, the non-aqueous solvent is a mixture of dichloromethane and methanol, wherein the weight ratio of dichloromethane to methanol is 1/1. In some embodiments of the spray-dried solid dispersion, Compound 1 is substantially amorphous.

In another aspect, provided herein is a pharmaceutical formulation comprising a spray-dried solid dispersion described herein, further comprising one or more pharmaceutical acceptable ingredients selected from the group consisting of one or more diluents, one or more disintegrants, one or more binders, one or more lubricants, one or more glidants, and one or more surfactants. In some embodiments, the one or more pharmaceutical acceptable ingredients are selected from the group consisting of microcrystalline cellulose, lactose monohydrate, croscarmellose sodium, magnesium stearate, colloidal silicon dioxide, mannitol, crospovidone, and sodium stearyl fumarate. In some embodiments, the one or more pharmaceutical acceptable ingredients are selected from the group consisting of microcrystalline cellulose, lactose monohydrate, croscarmellose sodium, magnesium stearate, and colloidal silicon dioxide. In some embodiments, the pharmaceutical formulation is in tablet form. In some embodiments, the pharmaceutical formulation is in capsule form.

### Methods of Dosing and Treatment Regimens

In one embodiment, Compound 1 described herein, or a pharmaceutically acceptable salt thereof, is used in the preparation of medicaments for the treatment of diseases or conditions in a mammal that would benefit from administration of a FXR agonist. Methods for treating any of the diseases or conditions described herein in a mammal in need of such treatment, involves administration of pharmaceutical compositions that include Compound 1 described herein, or a pharmaceutically acceptable salt, active metabolite, prodrug, or pharmaceutically acceptable solvate thereof, in therapeutically effective amounts to said mammal.

Disclosed herein, are methods of administering a FXR agonist in combination with an additional therapeutic agent. In some embodiments, the additional therapeutic agent comprises a therapeutic agent for treatment of diabetes or diabetes related disorder or conditions, alcoholic or non-alcoholic liver disease, inflammation related intestinal conditions, or cell proliferative disorders.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a Compound 1, or a pharmaceutically acceptable salt thereof, in order to prevent a return of the symptoms of the disease or condition.

In certain embodiments wherein the patient's condition does not improve, upon the doctor's discretion, the Compound 1 is administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for Compound 1 described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of Compound 1 described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of Compound 1, including further embodiments in which (i) the compound is administered once a day; or (ii) the compound is administered to the mammal multiple times over the span of one day.

In any of the aforementioned aspects are further embodiments comprising multiple administrations of the effective amount of Compound 1, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer Compound 1, or a pharmaceutically acceptable salt thereof, in combination with one or more other therapeutic agents.

In one embodiment, the therapeutic effectiveness of Compound 1 is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, Compound 1, or a pharmaceutically acceptable salt thereof, is co-administered with a second therapeutic agent, wherein Compound 1, or a pharmaceutically acceptable salt thereof, and the second therapeutic agent modulate different aspects of the disease, disorder or condition being treated, thereby providing a greater overall benefit than administration of either therapeutic agent alone.

### EXAMPLES

### List of Abbreviations

As used above, and throughout the description of the invention, the following abbreviations, unless otherwise indicated, shall be understood to have the following meanings:
ACN or MeCN acetonitrile
Bn benzyl
BOC or Boc tert-butyl carbamate
t-Bu tert-butyl
Cy cyclohexyl
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
equiv equivalent(s)
Et ethyl
Et₂O diethyl ether
EtOH ethanol
EtOAc ethyl acetate
HPLC high performance liquid chromatography
Me methyl
MeOH methanol
MS mass spectroscopy
NMR nuclear magnetic resonance
RP-HPLC reverse phase-high pressure liquid chromatography
TFA trifluoroacetic acid
THE tetrahydrofuran
TLC thin layer chromatography

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1: Preparation of Amorphous Compound 1

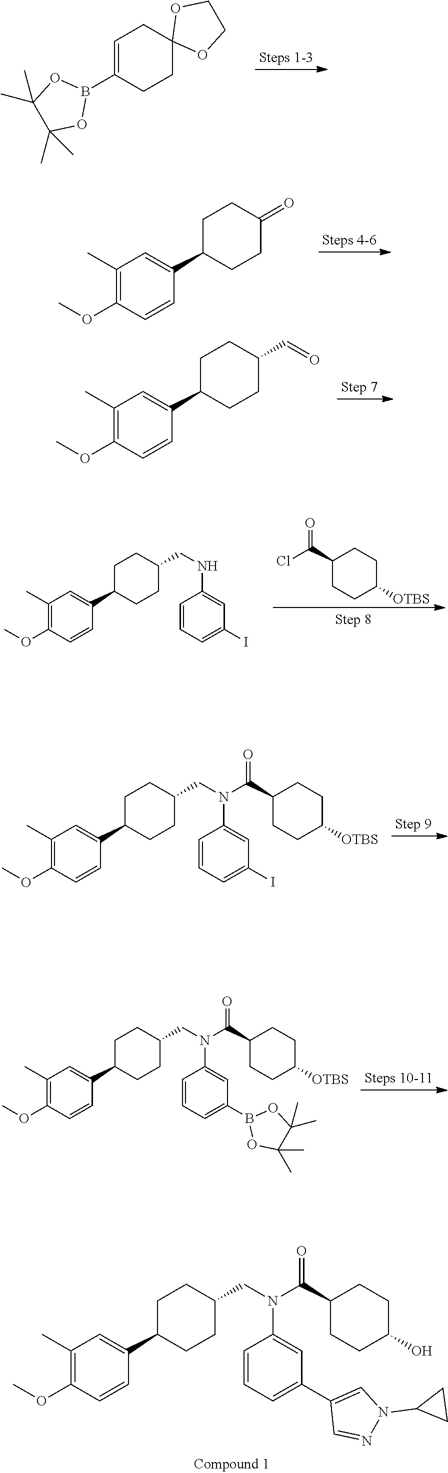

### Step 1: 8-(4-Methoxy-3-methylphenyl)-1,4-dioxaspiro[4.5]dec-7-ene

A mixture of 1,4-dioxa-spiro[4,5]dec-7-en-8-boronic acid pinacol ester (25.0 g, 93.9 mmol), 4-iodo-2-methylanisole (28.0 g, 113 mmol), 1,1′-bis(diphenylphosphino)ferrocene dichlorlopalladium(II) (1.38 g, 1.89 mmol), dioxane (470 mL) and 1 M Na₂CO₃ (282 mL, 282 mmol) was degassed with 3 vacuum/N₂ cycles, stirred at 50° C. for 2.5 h, and then allowed to cool to rt. The mixture was diluted with EtOAc (500 mL) and washed with sat'd NaHCO₃ (2×500 mL). The aqueous layers were back extracted with EtOAc (200 mL). The combined EtOAc extracts were dried (Na₂SO₄), filtered, concentrated and purified by silica gel chromatography (0-5% EtOAc in hexanes) to give 8-(4-methoxy-3-methylphenyl)-1,4-dioxaspiro[4.5]dec-7-ene (19.9 g, 81%). ¹H NMR (400 MHz, DMSO-d₆): δ 7.21-7.16 (m, 2H), 6.85 (d, 1H), 5.89-5.84 (m, 1H), 3.90 (s, 4H), 3.76 (s, 3H), 2.52-2.47 (m, 2H), 2.32 (br s, 2H), 2.13 (s, 3H), 1.77 (t, 2H); LCMS: 261.1 [M+H]⁺.

### Step 2: 8-(4-Methoxy-3-methylphenyl)-1,4-dioxaspiro[4.5]decane

Palladium on carbon (10 wt %, 8.08 g, 7.59 mmol) was added to a solution of 8-(4-methoxy-3-methylphenyl)-1,4-dioxaspiro[4.5]dec-7-ene (19.8 g, 76.1 mmol) in EtOAc (300 mL) at rt under N₂. The N₂ inlet was replaced with a balloon of H₂. The reaction was stirred for 4.5 h, filtered through Celite with EtOAc, and then concentrated to give 8-(4-methoxy-3-methylphenyl)-1,4-dioxaspiro[4.5]decane (18.2 g; contains 13% ketone) as a white solid. ¹H NMR (400 MHz, DMSO-d₆): δ 7.00-6.95 (m, 2H), 6.81 (d, 1H), 3.91-3.84 (m, 4H), 3.73 (s, 3H), 2.49-2.42 (m, 1H), 2.11 (s, 3H), 1.76-1.68 (m, 4H), 1.67-1.55 (m, 4H); LCMS: 263.1 [M+H]⁺.

### Step 3: 4-(4-Methoxy-3-methylphenyl)cyclohexanone

Formic acid (96%, 14 mL, 356 mmol) and then water (2.20 mL, 122 mmol) were added to a solution of 8-(4-methoxy-3-methylphenyl)-1,4-dioxaspiro[4.5]decane (18.2 g) in toluene (60 mL) at rt under N₂. The reaction was heated at 120° C. for 4 h, allowed to cool to rt, and then poured into 200 mL H₂O and 200 mL toluene. The toluene layer was washed with 200 mL H₂O and then 200 mL sat'd NaHCO₃. The aqueous layers were back extracted with 100 mL toluene. The combined toluene extracts were dried (Na₂SO₄), filtered and concentrated to give 4-(4-methoxy-3-methylphenyl)cyclohexanone (15.5 g, 88% over 2 steps) as a white solid. ¹H NMR (400 MHz, DMSO-d₆): δ 7.08-7.03 (m, 2H), 6.84 (d, 1H), 3.74 (s, 3H), 3.00-2.91 (m, 1H), 2.61-2.51 (m, 2H), 2.28-2.20 (m, 2H), 2.12 (s, 3H), 2.06-1.98 (m, 2H), 1.88-1.76 (m, 2H); LCMS: 219.0 [M+H]⁺.

### Step 4: 1-Methoxy-4-(4-(methoxymethylene)cyclohexyl)-2-methylbenzene

A mixture of (methoxymethyl)triphenylphosphonium chloride (35.74 g, 104.3 mmol) and THE (260 mL) under N₂ was cooled to −2.2° C. in an ice/brine bath. Sodium bis(trimethylsilyl)amide solution (2 M in THF, 50 mL, 100 mmol) was added dropwise via addition funnel over 12 min (internal temp≤0.6° C.) with THE rinsing (5 mL). The reaction was stirred for 30 min, and then 4-(4-methoxy-3-methylphenyl)cyclohexanone (14.5 g, 66.6 mmol) was added portionwise over 5 min (exotherm to 7.3° C.). Residual cyclohexanone was rinsed into the reaction with THE (20 mL). The reaction was stirred at 0° C. for 25 min, and then poured into 400 mL H₂O and 400 mL toluene. The toluene layer was washed with 400 mL H₂O, dried (Na₂SO₄), filtered, concentrated and purified by silica gel chromatography (0-5% EtOAc in hexanes) to give 1-methoxy-4-(4-(methoxymethylene)cyclohexyl)-2-methylbenzene (15.6 g, 95%) as a pale gold oil. ¹H NMR (400 MHz, DMSO-d₆): δ 6.99-6.94 (m, 2H), 6.80 (d, 1H), 5.87 (s, 1H), 3.73 (s, 3H), 3.48 (s, 3H), 2.78-2.71 (m, 1H), 2.56-2.44 (m, 1H), 2.10 (s, 3H), 2.17-2.09 (m, 1H), 2.01-1.91 (m, 1H), 1.83-1.73 (m, 2H), 1.72-1.63 (m, 1H), 1.38-1.23 (m, 2H); LCMS: 247.1 [M+H]⁺.

### Step 5: 4-(4-Methoxy-3-methylphenyl)cyclohexanecarbaldehyde

Formic acid (96%, 12.5 mL, 331 mmol) and then water (2.5 mL, 139 mmol) were added to a solution of 1-methoxy-4-(4-(methoxymethylene)cyclohexyl)-2-methylbenzene (16.05 g, 65.15 mmol) in toluene (130 mL) under N₂. The reaction was heated at 120° C. for 2 h, allowed to cool to rt, and then poured into 350 mL EtOAc and 350 mL H₂O. The organic layer was washed with 350 mL H₂O, dried (Na₂SO₄), filtered and concentrated to give 4-(4-methoxy-3-methylphenyl)cyclohexanecarbaldehyde (15.05 g) as a 1:1 mixture of stereoisomers.

### Step 6: trans-4-(4-Methoxy-3-methylphenyl)cyclohexanecarbaldehyde

Aqueous sodium hydroxide (3.2 M, 31 mL, 99 mmol) was added to the crude mixture from Step 5 (14.68 g, 63.19 mmoL), toluene (60 mL) and ethanol (250 mL) at rt. The reaction was stirred for 5.5 hours (equilibration monitored by NMR) and then poured into 350 mL H₂O and 350 mL EtOAc. The organic layer was washed with 350 mL H₂O, and the aqueous layers were back extracted with 150 mL EtOAc. The combined extracts were dried (Na₂SO₄), filtered, concentrated and purified by silica gel chromatography (0-5% EtOAc in hexanes) to give trans-4-(4-methoxy-3-methylphenyl)cyclohexanecarbaldehyde (10.17 g, 69%) as a white solid. ¹H NMR (400 MHz, DMSO-d₆): δ 9.60 (s, 1H), 7.01-6.97 (m, 2H), 6.82 (d, 1H), 3.74 (s, 3H), 2.41-2.27 (m, 2H), 2.12 (s, 3H), 2.03-1.96 (m, 2H), 1.87-1.80 (m, 2H), 1.51-1.39 (m, 2H), 1.35-1.23 (m, 2H); LCMS: 233.0 [M+H]⁺.

### Step 7: 3-Iodo-N-((trans-4-(4-methoxy-3-methylphenyl)cyclohexyl)methyl)aniline

Sodium triacetoxyborohydride (3.74 g, 17.6 mmol) was added to a solution of trans-4-(4-methoxy-3-methylphenyl)cyclohexanecarbaldehyde (2.56 g, 11.0 mmol), 3-iodoaniline (2.56 g, 11.7 mmol), acetic acid (1.3 mL, 23 mmol) and dichloroethane (45 mL) at rt under N₂. The reaction was stirred for 80 min, poured into 50 mL sat'd NaHCO₃ and extracted with 50 mL EtOAc. The EtOAc layer was washed with 50 mL sat'd NaHCO₃ and washed with 50 mL brine. The aqueous layers were combined and back extracted with 25 mL EtOAc. The combined organics were dried (Na₂SO₄), filtered, concentrated and purified by silica gel chromatography (0-5% EtOAc in hexanes) to give 3-iodo-N-((trans-4-(4-methoxy-3-methylphenyl)cyclohexyl)methyl)aniline (4.43 g, 88%) as a yellow oil. ¹H NMR (400 MHz, DMSO-d₆): δ 7.01-6.95 (m, 2H), 6.91 (s, 1H), 6.86-6.77 (m, 3H), 6.57 (d, 1H), 5.92 (t, 1H), 3.73 (s, 3H), 2.85 (t, 2H), 2.42-2.31 (m, 1H), 2.11 (s, 3H), 1.94-1.85 (m, 2H), 1.82-1.73 (m, 2H), 1.63-1.50 (m, 1H), 1.45-1.31 (m, 2H), 1.14-1.00 (m, 2H); LCMS: 436.4 [M+H]⁺.

### Step 8: trans-4-((tert-Butyldimethylsilyl)oxy)-N-(3-iodophenyl)-N-((trans-4-(4-methoxy-3-methylphenyl)cyclohexyl)methyl)cyclohexanecarboxamide

trans-4-((tert-Butyldimethylsilyl)oxy)cyclohexanecarbonyl chloride (74 mg/mL in toluene, 43 mL, 11.49 mmol) was added to a solution of 3-iodo-N-((trans-4-(4-methoxy-3-methylphenyl)cyclohexyl)methyl)aniline (3.32 g, 7.63 mmol), pyridine (2.5 mL, 31 mmol), and toluene (15 mL). The mixture was stirred at rt for 90 min, diluted with EtOAc (50 mL), and washed (50 mL H₂O, 50 mL sat'd NaHCO₃ and then 50 mL brine). The organic layer was dried (Na₂SO₄), filtered, concentrated, and purified by silica gel chromatography (0-10% EtOAc in hexanes) to give trans-4-((tert-butyldimethylsilyl)oxy)-N-(3-iodophenyl)-N-((trans-4-(4-methoxy-3-methylphenyl)cyclohexyl)methyl)cyclohexanecarboxamide (4.05 g, 79%) as a white foam. ¹H NMR (400 MHz, DMSO-d₆): δ 7.76 (d, 1H), 7.72 (s, 1H), 7.31 (d, 1H), 7.27 (t, 1H), 6.97-6.92 (m, 2H), 6.80-6.76 (m, 1H), 3.72 (s, 3H), 3.60-3.40 (m, 3H), 2.37-2.27 (m, 1H), 2.09 (s, 3H), 2.01-1.91 (m, 1H), 1.78-1.67 (m, 6H), 1.65-1.56 (m, 2H), 1.49-1.21 (m, 5H), 1.10-0.94 (m, 2H), 0.92-0.76 (m, 11H), −0.01 (s, 6H); LCMS: 676.6 [M+H]⁺.

### Step 9: trans-4-((tert-Butyldimethylsilyl)oxy)-N-((trans-4-(4-methoxy-3-methylphenyl)cyclohexyl)methyl)-N-(3-(4,4,5,5-tetramethyl-1,3,2-dioxaborolan-2-yl)phenyl)cyclohexanecarboxamide

A mixture of bis(pinacolato)diboron (1.42 g, 5.59 mmol), potassium acetate (1.45 g, 14.8 mmol), Pd(dppf)Cl₂ (135 mg, 0.18 mmol), and toluene (23 mL) was degassed with 3 vacuum/N₂ cycles. trans-4-((tert-butyldimethylsilyl)oxy)-N-(3-iodophenyl)-N-((trans-4-(4-methoxy-3-methylphenyl)cyclohexyl)methyl)cyclohexanecarboxamide (2.50 g, 3.70 mmol) was added to the mixture, and the reaction was degassed with 2 vacuum/N₂ cycles, heated at 115° C. for 3.5 h, and then allowed to cool to rt. The mixture was diluted with 75 mL EtOAc. The organics were washed with sat'd NaHCO₃ (2×75 mL), dried (Na₂SO₄), filtered, concentrated, and dried on high vacuum overnight to give trans-4-((tert-butyldimethylsilyl)oxy)-N-((trans-4-(4-methoxy-3-methylphenyl)cyclohexyl)methyl)-N-(3-(4,4,5,5-tetramethyl-1,3,2-dioxaborolan-2-yl)phenyl)cyclohexanecarboxamide (2.99 g, 120% crude product) as a brown solid. ¹H NMR (400 MHz, CDCl₃): δ 7.82-7.78 (m, 1H), 7.61-7.57 (m, 1H), 7.43 (t, 1H), 7.27-7.24 (m, 1H), 6.99-6.94 (m, 2H), 6.74 (d, 1H), 3.80 (s, 3H), 3.72-3.45 (m, 3H), 2.44-2.33 (m, 1H), 2.20 (s, 3H), 2.11-2.01 (m, 1H), 1.90-1.76 (m, 6H), 1.75-1.65 (m, 3H), 1.58-1.47 (m, 2H), 1.42-1.32 (m, 14H), 1.24-1.10 (m, 2H), 1.06-0.92 (m, 2H), 0.84 (s, 9H), 0.01 (s, 6H); LCMS: 676.6.

### Step 10: trans-4-((tert-Butyldimethylsilyl)oxy)-N-(3-(1-cyclopropyl-1H-pyrazol-4-yl)phenyl)-N-((trans-4-(4-methoxy-3-methylphenyl)cyclohexyl)methyl)cyclohexanecarboxamide

A mixture of 4-bromo-1-cyclopropyl-1H-pyrazole (65 mg, 0.35 mmol), trans-4-((tert-butyldimethylsilyl)oxy)-N-((trans-4-(4-methoxy-3-methylphenyl)cyclohexyl)methyl)-N-(3-(4,4,5,5-tetramethyl-1,3,2-dioxaborolan-2-yl)phenyl)cyclohexanecarboxamide (163 mg, 0.20 mmol), Cs₂CO₃ (196 mg, 0.60 mmol), Pd(dppf)Cl₂, DMF (2 mL), and H₂O (20 μL) was degassed with 3 vacuum/N₂ cycles, heated at 80° C. for 110 min, and allowed to cool to rt. The reaction was poured into 20 mL sat'd NaHCO₃ and then extracted with EtOAc (2×20 mL). The combined organics were washed with 20 mL brine, dried (Na₂SO₄), filtered, concentrated, and purified by silica gel chromatography (10-30% ethyl acetate in hexanes) to give trans-4-((tert-butyldimethylsilyl)oxy)-N-(3-(1-cyclopropyl-1H-pyrazol-4-yl)phenyl)-N-((trans-4-(4-methoxy-3-methylphenyl)cyclohexyl)methyl)cyclohexanecarboxamide (63 mg, 48%) as a white foam. ¹H NMR (400 MHz, DMSO-d₆): δ 8.34 (s, 1H), 7.93 (s, 1H), 7.61 (d, 1H), 7.56-7.52 (m, 1H), 7.43 (t, 1H), 7.08 (d, 1H), 6.98-6.91 (m, 2H), 6.81-6.75 (m, 1H), 3.77-3.69 (m, 4H), 3.64-3.45 (m, 3H), 2.38-2.28 (m, 1H), 2.13-2.02 (m, 4H), 1.81-1.68 (m, 6H), 1.68-1.59 (m, 2H), 1.51-1.36 (m, 3H), 1.36-1.22 (m, 2H), 1.12-0.95 (m, 6H), 0.89-0.74 (m, 11H), −0.03 (s, 6H); LCMS: 656.6 [M+H]⁺.

### Step 11: trans-N-(3-(1-Cyclopropyl-1H-pyrazol-4-yl)phenyl)-4-hydroxy-N-((trans-4-(4-methoxy-3-methylphenyl)cyclohexyl)methyl)cyclohexanecarboxamide (Compound 1)

Aqueous hydrochloric acid (6 N, 0.13 mL, 0.78 mmol) was added to a solution of trans-4-((tert-butyldimethylsilyl)oxy)-N-(3-(1-cyclopropyl-1H-pyrazol-4-yl)phenyl)-N-((trans-4-(4-methoxy-3-methylphenyl)cyclohexyl)methyl)cyclohexanecarboxamide (62 mg, 0.095 mmol), methanol (0.5 mL) and tetrahydrofuran (0.5 mL) at 0° C. The reaction was allowed to warm to rt, stirred for 40 min, poured into 20 mL cold sat'd NaHCO₃, and then extracted with EtOAc. The organics were washed with 20 mL sat'd NaHCO₃ and washed with 20 mL brine. The first aqueous wash was back extracted with 20 mL EtOAc. The combined extracts were dried (Na₂SO₄), filtered, concentrated, and purified by silica gel chromatography (0-7% MeOH in DCM) to give trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide (Compound 1) (50 mg, 98%) as an off-white foam. ¹H NMR (400 MHz, DMSO-d₆): δ 8.34 (s, 1H), 7.94 (s, 1H), 7.60 (d, 1H), 7.56-7.51 (m, 1H), 7.44 (t, 1H), 7.09 (d, 1H), 6.98-6.92 (m, 2H), 6.81-6.75 (m, 1H), 4.39 (d, 1H), 3.78-3.69 (m, 4H), 3.63-3.48 (m, 2H), 3.30-3.20 (m, 1H), 2.38-2.28 (m, 1H), 2.09 (s, 3H), 2.08-1.99 (m, 1H), 1.80-1.68 (m, 6H), 1.67-1.58 (m, 2H), 1.48-1.37 (m, 3H), 1.32-1.20 (m, 2H), 1.11-0.95 (m, 6H), 0.81-0.67 (m, 2H); LCMS: 542.5 [M+H]⁺.

### Example 2: Preparation of Crystalline Compound 1 (Form 1 by Maturation in Ethanol)

Amorphous Compound 1 (750 mg) was suspended in ethanol (5 mL) and slurried at room temperature for 2.5 hours. The resulting solid was filtered and analysed by XRPD which showed it to be amorphous. The amorphous solid was re-suspended in ethanol (5 mL) and placed in a maturation chamber to cycle between room temperature and 50° C. for 5 days (4 hours at each temperature). The resulting solid was filtered by suction and vacuum dried overnight to afford crystalline Compound 1, Form 1.

### Example 3: Preparation of Compound 1 (Form 1B)

Amorphous Compound 1 (ca. 500 mg) was weighed out and suspended in THF (10 vol) yielding a turbid solution. Additional amorphous Compound 1 (100 mg) was added and a thicker suspension was observed. Maturation at 25° C. was carried out for four days. A white suspension was observed, filtered by gravity and air-dried. The solid was analysed wet. Further, an aliquot was taken and dried under vacuum overnight at 25° C. This material was also characterised.

### Example 4: Preparation of Compound 1 (Form 1C)

Amorphous Compound 1 (ca. 500 mg) was weighed out and dissolved in 1,4-dioxane (15 vol) at 50° C. Heptane (10 vol) was added at room temperature, yielding a turbid solution, which was placed at 4° C. overnight. A white suspension was observed, filtered by gravity and air-dried. The solid was analysed wet. Further, an aliquot was taken and dried under vacuum overnight at 25° C. This material was also characterised.

### II. Characterization of Compounds

### Example 5: X-ray Powder Diffraction (XRPD)

X-Ray Powder Diffraction patterns were collected on a Bruker D8 diffractometer using Cu Kα radiation (40 kV, 40 mA), 0-20 goniometer, and divergence of V4 and receiving slits, a Ge monochromator and a Lynxeye detector. The instrument was performance checked using a certified Corundum standard (NIST 1976). The software used for data collection was Diffrac Plus XRD Commander v2.6.1 and the data were analysed and presented using Diffrac Plus EVA v15.0.0.0.

Samples were run under ambient conditions as flat plate specimens using powder as received. The sample was gently packed into a cavity cut into polished, zero-background (510) silicon wafer. The sample was rotated in its own plane during analysis. The parameters for data collection were: angular range, 2 to 42° 2θ; step size, 0.05° 2θ; collection time, 0.5 s/step.

XRPD analysis (FIG. 1) of Form 1 of Compound 1 showed Form 1 of the free base to be crystalline. The corresponding peak list is shown in the Table below:

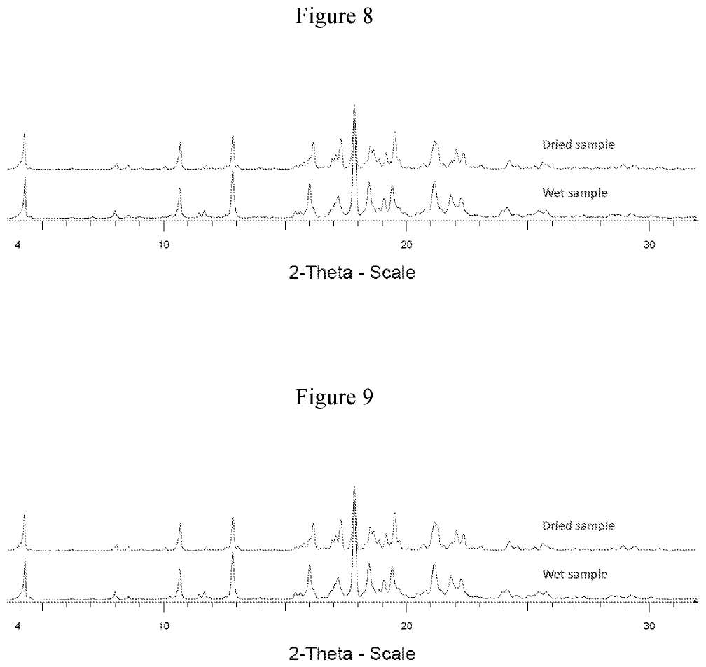

XRPD analysis (FIG. 8) of Form 1B of Compound 1 showed Form 1B of the free base to be crystalline. The Form 1B XRPD scan displays the main features of Form 1, but showed slight shifts at higher 2θ angles.

XRPD analysis (FIG. 9) of Form 1C of Compound 1 showed Form 1C of the free base to be crystalline. The Form 1C XRPD scan displays the main features of Form 1, but showed slight shifts at higher 2θ angles.

### Example 6: Single Crystal X-Ray Diffraction

Data were collected on an Oxford Diffraction Supernova Dual Source, Cu at Zero, Atlas CCD diffractometer equipped with an Oxford Cryosystems Cobra cooling device. The data were collected using CuKα radiation. Structures were typically solved using either the SHELXS or SHELXD programs and refined with the SHELXL program as part of the Bruker AXS SHELXTL suite (V6.10). Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter.

A single crystal structure of Form 1 of Compound 1 was collected using crystals from an emulsion formulation. Form 1 of Compound 1 crystallizes in the triclinic system, space group P-1 with the final R1 [I>2σ(I)]=6.17%. The structural data show that Form 1 of Compound 1 is the trans-trans isomer. Crystal data are summarized in the Table 1 below:

TABLE 1
Crystal data for Form 1 of Compound 1

| Empirical formula | C₃₄H₄₃N₃O₃ | |
|---|---|---|
| Formula weight | 541.71 | |
| Temperature | 100(2) K | |
| Wavelength | 1.54178 Å | |
| Crystal size | 0.120 × 0.070 × 0.040 mm | |
| Crystal habit | colorless plate | |
| Crystal system | Triclinic | |
| Space group | P-1 | |
| Unit cell dimensions | a = 11.3510(6) Å | α = 88.704(5)° |
| | b = 13.2316(7) Å | β = 76.176(5)° |
| | c = 20.3861(13) Å | γ = 89.628(4)° |

| | |
|---|---|
| Volume | 2972.4(3) Å³ |
| Z | 4 |
| Density (calculated) | 1.211 Mg/m³ |
| Absorption coefficient | 0.608 mm⁻¹ |
| F(000) | 1168 |

Data collection and structure refinement are summarized in Table 2 below:

TABLE 2
Data collection and structure refinement for Form 1 of Compound 1

| | |
|---|---|
| Radiation source | SuperNova (Cu) X-ray Source, CuKα |
| Data collection method | scans |
| Theta range for data collection | 6.688 to 66.599° |
| Index ranges | −13 ≤ h ≤ 13, −14 ≤ k ≤ 15, −24 ≤ l ≤ 24 |
| Reflections collected | 42372 |
| Independent reflections | 10501 [R(int) = 0.0692] |
| Coverage of independent reflections | 97.7% |
| Variation in check reflections | n/a |
| Absorption correction | Semi-empirical from equivalents |
| Max. and min. transmission | 1.00000 and 0.88917 |
| Structure solution technique | Direct Methods |
| Structure solution program | SHELXTL (Sheldrick, 2013) |
| Refinement technique | Full-matrix least-squares on F² |
| Refinement program | SHELXL-2013 (Sheldrick, 2013) |
| Function minimized | Σw(Fₒ² − F_{c}²)² |
| Data/restraints/parameters | 10501/2347/945 |
| Goodness-of-fit on F² | 0.949 |
| Δ/σₘₐₓ | 0.000 |
| Final R indices | |
| 7381 data; I > 2σ(I) | R1 = 0.0617, wR2 = 0.1509 |
| all data | R1 = 0.0915, wR2 = 0.1750 |
| Weighting scheme | w = 1/[σ² (Fₒ²) + (0.0728P)² + 2.0608P] |
| | where P = (Fₒ² + 2F_{c}²)/3 |
| Extinction coefficient | n/a |
| Largest diff. peak and hole | 0.797 and −0.837 eÅ⁻³ |

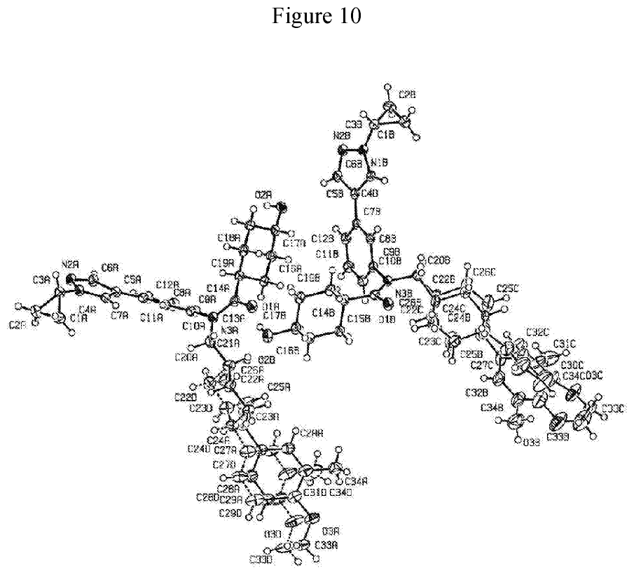

The asymmetric unit shows two molecules of Form 1 of Compound 1 which both exhibit regions of disorder. The asymmetric unit shows two molecules of Form 1 of Compound 1 which both exhibit regions of disorder (FIG. 10).

### Example 7: Polarized Light Microscopy (PLM)

The presence of crystallinity (birefringence) was determined using an Leica LM/DM or Nikon SMZ1500 polarising microscope, equipped with a digital video camera for image capture. A small amount of each sample was placed on a glass slide, mounted in immersion oil and covered with a glass slip, the individual particles being separated as well as possible. The sample was viewed with appropriate magnification and partially polarised light, coupled to a λ false-colour filter.

PLM analysis of Form 1 of Compound 1 showed agglomerated prismatic crystals, sizes up to 150 μm (length).

### Example 8: Thermo-gravimetric Analysis (TGA)

TGA data were collected on a TA Instruments Q500 TGA, equipped with a 16 position auto-sampler. The instrument was temperature calibrated using certified Alumel and Nickel. Typically 5-10 mg of each sample was loaded onto a pre-tared aluminium DSC pan and heated at 10° C./min from ambient temperature to 350° C. A nitrogen purge at 60 mL/min was maintained over the sample.

The instrument control software was Advantage for Q Series v2.5.0.256 and Thermal Advantage v5.5.3 and the data were analysed using Universal Analysis v4.5A.

TGA (FIG. 2) of Form 1 of Compound 1 showed no weight loss before decomposition (with onset at about 300° C.).

### Example 9: Differential Scanning Calorimetry (DSC)

DSC data were collected on a TA Instruments Q2000 equipped with a 50 position auto-sampler. The calibration for thermal capacity was carried out using sapphire and the calibration for energy and temperature was carried out using certified indium. Typically 0.5-3 mg of each sample, in a pin-holed aluminium pan, was heated at 10° C./min from 25° C. to 350° C. A purge of dry nitrogen at 50 ml/min was maintained over the sample.

The instrument control software was Advantage for Q Series v2.8.0.394 and Thermal Advantage v5.5.3 and the data were analysed using Universal Analysis v4.5A.

DSC analysis (FIG. 3) of Form 1 of Compound 1 showed a sharp melting endotherm with onset at 178.2° C. (95 J/g).

### Example 10: Gravimetric Vapor Sorption (GVS)

Sorption isotherms were obtained using a SMS DVS Intrinsic moisture sorption analyser, controlled by DVS Intrinsic Control software v1.0.1.2 (or v1.0.1.3). The sample temperature was maintained at 25° C. by the instrument controls. The humidity was controlled by mixing streams of dry and wet nitrogen, with a total flow rate of 200 ml/min The relative humidity was measured by a calibrated Rotronic probe (dynamic range of 1.0-100% RH), located near the sample. The weight change, (mass relaxation) of the sample as a function of % RH was constantly monitored by the microbalance (accuracy ±0.005 mg).

Typically 5-20 mg of sample was placed in a tared mesh stainless steel basket under ambient conditions. The sample was loaded and unloaded at 40% RH and 25° C. (typical room conditions). A moisture sorption isotherm was performed as outlined below (2 scans giving 1 complete cycle). The standard isotherm was performed at 25° C. at 10% RH intervals over a 0-90% RH range. Data analysis was carried out using Microsoft Excel using DVS Analysis Suite v6.2 (or v6.1 or v6.0).

TABLE 3
Method for SMS DVS Intrinsic experiments

| Parameter | Value |
|---|---|
| Adsorption - Scan 1 | 40-90 |
| Desorption/Adsorption - Scan 2 | 90-0, 0-40 |
| Intervals (% RH) | 10 |
| Number of Scans | 2 |
| Flow rate (ml/min) | 200 |
| Temperature (° C.) | 25 |
| Stability (° C./min) | 0.2 |
| Sorption Time (hours) | 6 hour time out |

The sample was recovered after completion of the isotherm and re-analysed by XRPD.

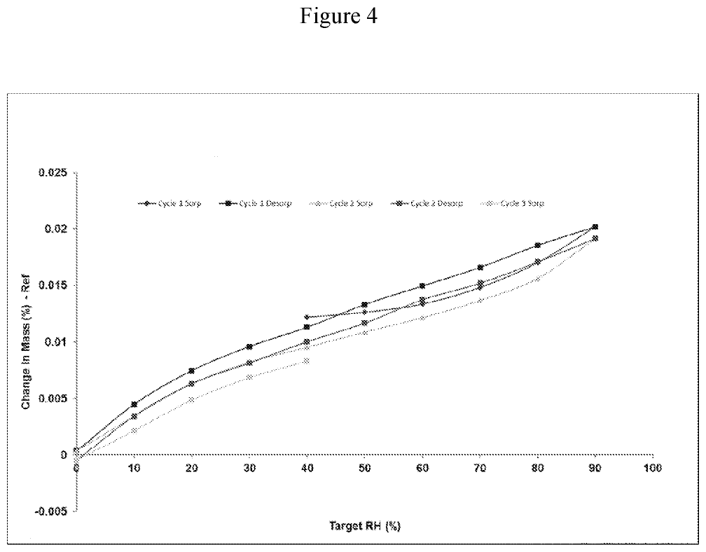

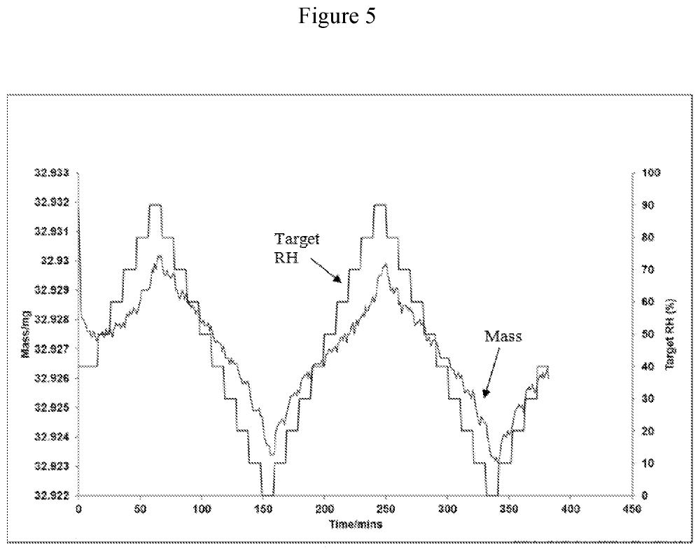

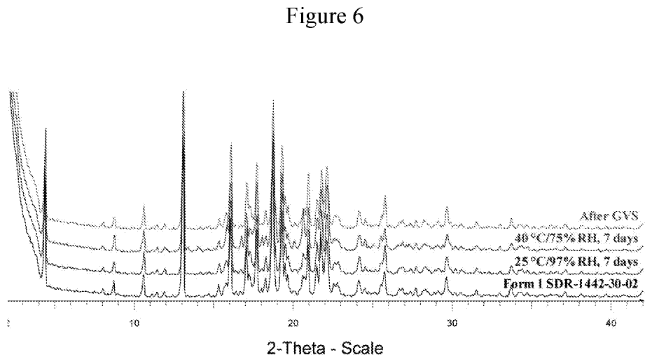

GVS analysis (FIGS. 4 and 5) of Form 1 of Compound 1 showed 0.02% moisture uptake between 0-90% RH. Post-GVS analysis by XRPD (FIG. 6, upper trace (d)) showed no change.

### Example 11: High Performance Liquid Chromatography-Ultraviolet Detection (HPLC-UV)

Purity analysis was performed on an Agilent HP1100 series system equipped with a diode array detector and using ChemStation software vB.04.03 using the method detailed in Table 4 below:

TABLE 4
HPLC method for chemical purity determinations

| Parameter | Value | | |
|---|---|---|---|
| Type of method | Reverse phase with gradient elution | | |
| Sample Preparation | 0.2 mg/ml in acetonitrile : water 1:1 | | |
| Column | Supelco Ascentis Express C18, 100 × 4.6mm, 2.7 μm | | |
| Column Temperature (° C.) | 25 | | |
| Injection (μl) | 2 | | |
| Wavelength, Bandwidth (nm) | 255, 90 | | |
| Flow Rate (ml/min) | 2 | | |
| Phase A | 0.1% TFA in water | | |
| Phase B | 0.085% TFA in acetonitrile | | |
| | Time (min) | % Phase A | % Phase B |
| Timetable | 0 | 95 | 5 |
| | 6 | 5 | 95 |
| | 6.2 | 95 | 5 |
| | 8 | 95 | 5 |

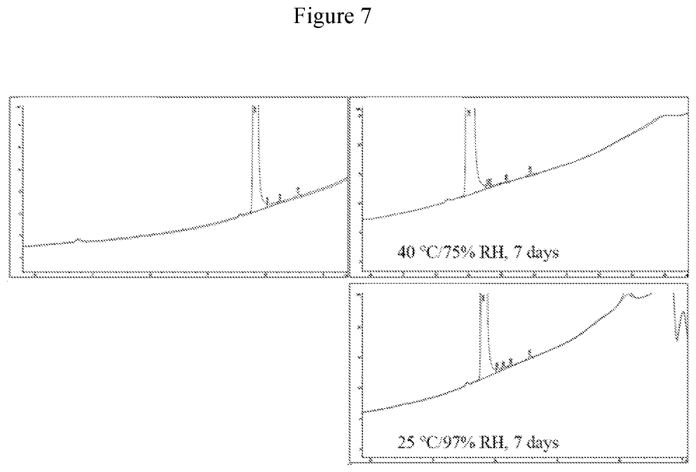

HPLC purity of Form 1 of Compound 1 as prepared by maturation in ethanol was measure to be 99.45% area under the curve (AUC) (see FIG. 7, upper left; compared with 98.9% AUC of amorphous Compound 1, as-synthesized).

HPLC purity of Form 1 of Compound 1 as prepared by maturation in ethanol and after 7 days at 25° C. and 97% RH was measured to be 99.40% AUC (see FIG. 7, lower right). Further, Form 1 of Compound 1 was unchanged by XRPD after 7 days at 25° C. and 97% RH (see FIG. 6, trace (b)).

HPLC purity of Form 1 of Compound 1 as prepared by maturation in ethanol and after 7 days at 40° C. and 75% RH was measured to be 99.48% AUC (see FIG. 7, upper right). Further, Form 1 of Compound 1 was unchanged by XRPD after 7 days at 40° C. and 75% RH (see FIG. 6, trace (c)).

These results and data showed Form 1 of Compound 1 to be an anhydrous, highly pure, non-hygroscopic highly crystalline material. The observed melting endotherm had an onset at 178.2° C. The material is composed of agglomerated prismatic crystals of varying sizes up to 150 μm in length. No significant moisture uptake was observed by GVS and no changes by XRPD were seen after storage for 7 days at 40° C./75% RH and 25° C./97% RH.

### III. Polymorphism Study

### Example 12: Solvents

A series of 24 Class 2 and 3 solvents, and solvent mixtures, listed in Table 5 below, were used for a polymorphism study.

TABLE 5
Solvent selection for the polymorphism study

| No. | Solvent | ICH Class |
|---|---|---|
| 1 | n-Heptane | 3 |
| 2 | 1,4-Dioxane | 2 |
| 3 | Methanol | 2 |
| 4 | Toluene | 2 |
| 5 | Cumene | 3 |
| 6 | Acetone | 3 |
| 7 | Anisole | 3 |
| 8 | Dichloromethane | 2 |
| 9 | Tetrahydrofuran | 2 |
| 10 | N,N-Dimethylacetamide | 2 |
| 11 | 2-Propanol | 3 |
| 12 | Ethanol | 3 |
| 13 | Ethyl Acetate | 3 |
| 14 | Water | 3 |
| 15 | Isopropyl Acetate | 3 |
| 16 | Nitromethane | 2 |
| 17 | Acetonitrile | 2 |
| 18 | Dimethyl Sulfoxide | 3 |
| 19 | Methyl tert-Butyl Ether | 3 |
| 20 | Methylisobutyl Ketone | 3 |
| 21 | Dimethoxyethane | 2 |
| 22 | 10% Water/EtOH | 3 |
| 23 | 10% Water/IPA | 3 |
| 24 | 10% Water/THF | 2 |

### Example 13: Maturation at Room Temperature

Amorphous Compound 1 (ca. 40 mg) was suspended in each of the 24 selected solvents and solvent mixtures at room temperature. The suspensions were subjected to maturation at room temperature for two days. Several solutions observed were stored at 4° C. overnight. If solutions were still observed, the vials were uncapped to allow for evaporation. Any solids were analysed by XRPD.

### Example 14: Maturation Using Temperature Cycles

Amorphous Compound 1 (ca. 25 mg) was suspended in each of the 24 selected solvents and solvent mixtures at 50° C. Maturation cycles were between 50° C.-room temperature, 4 hours at each temperature. Aliquots were filtered after 24 hours of cycling and analysed by XRPD. Several solutions observed were stored at 4° C. overnight. If solutions were still observed, the vials were uncapped to allow for evaporation. Any solids were analysed by XRPD.

### Example 15: Maturation at 5° C.

Amorphous Compound 1 (ca. 25 mg) was suspended in each of the 24 selected solvents and solvent mixtures at 5° C. (with the exception of 1,4-dioxane and DMSO, which freeze at this temperature, these were added at room temperature). The suspensions were subjected to maturation at 5° C. for two days. Where solutions were observed, slow evaporation was set up. Any solids were analysed by XRPD.

### Example 16: Anti-Solvent Additions

Amorphous Compound 1 (ca. 25 mg) was dissolved in four selected solvents (DCM, THF, 1,4-dioxane and DMSO; 15 volumes) at 50° C. Dissolution was not possible in methanol (15 volumes), where light suspensions were observed. Each solution or light suspension was treated with anti-solvent at room temperature until turbidity was observed or a maximum of 20 volumes was added. The resulting solutions, turbid solutions and suspensions were stored at 4° C. in a refrigerator for five days. Any solutions still observed were opened for evaporation. Any solids observed were analysed by XRPD.

### Example 17: Cooling and Evaporation Screen

Dissolution of amorphous Compound 1 (ca. 25 mg) in the 24 selected solvents (15 volumes) was attempted at 50° C. Maturation at 50° C. was carried out overnight. Any solutions observed were placed in fridge (4° C.) for five days and later opened for evaporation if they were still solutions. For any suspensions, an aliquot was filtered and the saturated mother liquors were placed in a refrigerator at 4° C. for five days. The remaining suspensions were matured at 50° C. for six days. Any solids observed were analysed by XRPD.

### Results of Polymorphism Study Examples 12-17

In the maturation screens summarized in Examples 12-17, above, the majority of suspensions observed became slightly “thicker” a few minutes after the addition of the solvent, suggesting crystallisation occurred fairly quickly.

Most solids analysed showed the crystalline Form 1. In a few instances, the high throughput raw XRPD data showed slight differences, such as split peaks and/or peak shifts.

### IV. Compound 1 Spray Dried Dispersions

### Example 18: Compound 1/Polymer Combinations Screen

A polymeric based spray dried dispersion for Compound 1 was developed. Several Compound 1/polymer combinations were screened and evaluated using computational models. The evaluated polymers were PVP/VA 64, PVP 30, HPMC-AS M, HPMCAS-L, Eudragit L100-55, Eudragit L100, Eudragit EPO, HPMC E15, HPMC E3, HPMCP-HP55, PVA, and Soluplus. The Compound 1/polymer combinations were evaluated for: 1) Miscibility assessment—In silico simulations to assess phase separation propensity with different stabilizing carriers and drug loadings; 2) API/Polymer solubility confirmation—for each lead condition, a series of compatible solvent systems were tested; 3) Solvent casting—Solvent casting trials with different stabilizing carriers and drug loadings to further narrow the formulation variables; and 4) Supersautration studies—Evaluation of the precipitation inhibition of different stabilizing carriers using a solvent-shift method. Based on the screening studies Compound 1 and Eudragit L100 50% (w/w) and Compound 1 and PVP/VA 64 50% (w/w) were scaled up.

### Example 19: Compound 1/Polymer Combination Lab-Scale Prototype Manufacturing

Spray drying. A lab-scale spray drier (Buchi B-290 Spray Dryer), was used to dry the feed solution. The unit was equipped with a two fluid nozzle with a nozzle tip and cap 0.7 and 1.5 mm, respectively. The spray-drying unit was operated with nitrogen in open-loop configuration (i.e., without recirculation of the drying nitrogen) and the aspirator blowing at 100% capacity.

Secondary drying. A lab-scale vacuum tray dryer was used to reduce the residual solvents content of the wet spray dried dispersions. Secondary drying was carried out for 48 hours at 50° C. under vacuum and with nitrogen sweep.

Solution preparation. Solutions for prototype manufacturing of a spray dried dispersion using Eudragit L100 or PVP/VA 64 were prepared according to the following general procedure: the total amount of solvent was charged to an empty vessel; the total amount of polymer was slowly added under stirring; stirring was continued until the polymer was completely dissolved; the total amount of Compound 1 was slowly added under stirring; and stirring was continued until Compound 1 was completely dissolved. Representative solutions of Compound 1 and Eudragit L100, and Compound 1 and PVP/VA 64 were prepared according to the quantities and ratios in Table 6 below (where “C_feed”=solids content in the feed mixture [% w/w], and “C_Compound 1”=Compound 1 content in the feed mixture [% w/w]):

TABLE 6
Solutions for prototype manufacture of spray dried dispersions

| | | Compound 1/ | Compound 1/ |
|---|---|---|---|
| Quantities and Ratios | | (Eudragit L100) | (PVP/VA 64) |
| Compound 1 | g | 75 | 75 |
| Eudragit L100 | g | 75 | — |
| PVP/VA 64 | g | — | 75 |
| Methylene chloride | g | 945 | 1080 |
| Methanol | g | 405 | 270 |
| Total solids | g | 150 | 150 |
| Total liquids | g | 1350 | 1350 |
| Compound 1 load | % w/w | 50 | 50 |
| C_feed | % w/w | 10 | 10 |
| C_Coumpound 1 | % w/w | 5 | 5 |

### Results

The main process data and analytical results are summarized in Table 7 below (where “T_feed”=temperature of the feed solution [° C.], “F_drying”=flow rate of drying gas in the spray drier [kg/h], “F_atom”=atomization gas flow rate [g/min], “T_out”=drying gas temperature at the outlet of the drying chamber [° C.], “F_feed”=flow rate of feed solution to the spray dryer [kg/h], “GC”=gas chromatography, “KF”=Karl Fisher, “TFN”=two fluid nozzle, and “PSD”=particle size distribution):

TABLE 7
Main process data and analytical results from prototype manufacture of spray dried dispersions
Feed solution parameters

| | | | |
|---|---|---|---|
| Formulation | | Compound 1:Eudragit L100 | Compound 1:PVP/VA 64 |
| T_feed | ° C. | room temperature | room temperature |

Spray drying parameters

| | | | |
|---|---|---|---|
| Nozzle (orifice/core) | mm | TFN (0.7/1.5) | TFN (0.7/1.5) |
| F_drying | kg/h | 40 | 40 |
| F_atom | kg/h | 2.15 | 2.15 |
| T_out | ° C. | 40 | 40 |
| F_feed | kg/h | 0.44 | 0.44 |

Process throughput and yield

| | | | |
|---|---|---|---|
| Drying time | hh:mm | 03:25 | 03:25 |
| Yield (wet basis) | g | 135.9 | 124.1 |
| Yield (wet basis) | % w/w | 90.6 | 82.7 |

Secondary drying conditions

| | | | |
|---|---|---|---|
| Vacuum | — | yes | yes |
| Temperature | ° C. | 50 | 50 |
| Drying time | h | 72 | 72 |

Analytical results after secondary drying

| | | | |
|---|---|---|---|
| Residual solvents content by TGA | % w/w | 1.9 | 1.5 |
| Methylene chloride by GC | ppm | 5.1 | 5.5 |
| Methanol by GC | ppm | 130.7 | 18.6 |
| Water by KF | % w/w | 2.4 | 1.2 |
| Description | | White to off white solid | White to off white solid |
| XRPD | — | Amorphous form | Amorphous form |
| PSD Dᵥ₁₀ | μm | 1.0 | 1.2 |
| PSD Dᵥ₅₀ | μm | 7.0 | 4.7 |
| PSD Dᵥ₉₀ | μm | 17.4 | 10.8 |

Both spray dried dispersions (Compound 1: Eudragit L100 and Compound 1: PVP/VA 64) were amorphous after secondary drying, as indicated by the absence of crystalline peaks (XRPD) and melting endotherms characteristic of crystalline material (DSC).

### Example 20: Compound 1 Spray Dried Dispersions Stability Study

The two Compound 1 spray dried dispersions (Compound 1: Eudragit L100 and Compound 1: PVP/VA 64) were stored for 1 month in open vials at 40° C./75% RH. No chemical degradation was observed for either spray dried dispersion. In addition, the amorphous state for each spray dried dispersion was maintained.

### V. Compound 1 FXR Activity

### Example 21: In Vitro FXR Assay (TI)

### Seeding

CV-1 cells were seeded at a density of 2,000,000 cells in a T175 flask with DMEM+10% charcoal double-stripped FBS and incubated at 37° C. in 5% CO₂ for 18 h (O/N).

### Transfection

After 18 h of incubation, the medium in the T175 flask was changed with fresh DMEM+10% charcoal super-stripped serum. In a polypropylene tube, 2500 μL OptiMEM (Life Technologies, Cat #31985-062) was combined with expression plasmids for hFXR, hRXR, TK-ECRE-luc and pCMX-YFP. The tube was then briefly vortexed and incubated at room temperature for 5 minutes. Transfection reagent (X-tremeGENE HP from Roche, Cat #06 366 236 001) was added to the OptiMEM/plasmid mixture vortexed and incubated at room temperature for 20 minutes. Following incubation, the transfection reagent/DNA mixture complex was added to cells in the T175 flask and the cells were incubated at 37° C. in 5% CO₂ for 18 h (O/N).

### Addition of Compound 1

Compound 1 was serially diluted in DMSO and added to transfected CV-1 cells. The cells were then incubated for 18 hrs. The next day cells were lysed and examined for luminescence. Compound 1 TK hFXR: EC₅₀≤0.25 uM.

We claim:

1. A crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt or solvate thereof.

2. The crystalline form of claim 1, wherein the trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide is a free base.

3. The crystalline form of claim 2, wherein the crystalline form of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide has at least one of the following properties:
(a) an X-ray powder diffraction (XRPD) pattern substantially the same as shown in FIG. 1;
(b) an X-ray powder diffraction (XRPD) pattern with characteristic peaks at 4.4° 2-Theta, 13.0° 2-Theta, 16.0° 2-Theta, 17.0° 2-Theta, 17.7° 2-Theta, 18.7° 2-Theta, 19.3° 2-Theta, 20.9° 2-Theta, 21.7° 2-Theta, and 22.1° 2-Theta;
(c) a thermo-gravimetric analysis (TGA) substantially similar to the one set forth in FIG. 2;
(d) a DSC thermogram substantially similar to the one set forth in FIG. 3;
(e) a DSC thermogram with an endotherm having an onset at about 178° C.;
(f) non-hygroscopicity; or
(g) combinations thereof.

4. The crystalline form of claim 3, wherein the crystalline form has an X-ray powder diffraction (XRPD) pattern substantially the same as shown in FIG. 1.

5. The crystalline form of claim 3, wherein the crystalline form has an X-ray powder diffraction (XRPD) pattern with characteristic peaks at 4.4° 2-Theta, 13.0° 2-Theta, 16.0° 2-Theta, 17.0° 2-Theta, 17.7° 2-Theta, 18.7° 2-Theta, 19.3° 2-Theta, 20.9° 2-Theta, 21.7° 2-Theta, and 22.1° 2-Theta.

6. The crystalline form of claim 3, wherein the crystalline form has a thermo-gravimetric analysis (TGA) substantially similar to the one set forth in FIG. 2.

7. The crystalline form of claim 3, wherein the crystalline form has a DSC thermogram substantially similar to the one set forth in FIG. 3.

8. The crystalline form of claim 3, wherein the crystalline form has a DSC thermogram with an endotherm having an onset at about 178° C.

9. The crystalline form of claim 3, wherein the crystalline form is non-hygroscopic.

10. The crystalline form of claim 3, wherein the crystalline form is characterized as having properties (a), (b), (c), (d), (e), and (f).

11. The crystalline form of any one of claims 3-10, wherein the crystalline form is obtained from acetone, acetonitrile, anisole, methyl t-butyl ether, dimethoxyethane, 1,4-dioxane, ethanol, ethyl acetate, isopropyl acetate, methanol, methanol/water, ethanol/water, nitromethane, methyl isobutyl ketone, 2-propanol, 2-propanol/water, tetrahydrofuran, tetrahydrofuran/water, tetrahydrofuran/methyl t-butyl ether, toluene, water, heptane, or cumene, or combinations thereof.

12. The crystalline form of any one of claims 3-11, wherein the crystalline form is obtained from ethanol.

13. The crystalline form of any one of claims 3-11, wherein the crystalline form is obtained from tetrahydrofuran/methyl t-butyl ether.

14. The crystalline form of any one of claims 3-13, wherein the crystalline form is unsolvated.

15. The crystalline form of any one of claims 3-14, wherein the crystalline form is anhydrous.

16. A pharmaceutical composition comprising the crystalline form of any one of claims 1-15, or a pharmaceutically acceptable salt, or solvate thereof, and at least one inactive ingredient selected from pharmaceutically acceptable carriers, diluents, and excipients.

17. The crystalline form of any one of any one of claims 1-15 for use in medicine.

18. A spray-dried solid dispersion, comprising: (a) trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide, and (b) a pharmaceutically acceptable polymer; wherein trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide is dispersed in a polymer matrix formed from the pharmaceutically acceptable polymer.

19. The spray-dried solid dispersion of claim 18, wherein the pharmaceutically acceptable polymer is selected from PVP/VA 64, PVP 30, HPMC-AS M, HPMCAS-L, Eudragit L100-55, Eudragit L100, Eudragit EPO, HPMC E15, HPMC E3, HPMCP-HP55, PVA, and Soluplus.

20. The spray-dried solid dispersion of claim 18 or claim 19, wherein the pharmaceutically acceptable polymer is selected from PVP/VA 64, PVP 30, HPMC-AS M, Eudragit L100-55, Eudragit L100, and HPMC E15.

21. The spray-dried solid dispersion of any one of claims 18-20, wherein the pharmaceutically acceptable polymer is Eudragit L100.

22. The spray-dried solid dispersion of any one of claims 18-20, wherein the pharmaceutically acceptable polymer is PVP/VA 64.

23. The spray-dried solid dispersion of any one of claims 18-22, wherein the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is from 9:1 to 1:9.

24. The spray-dried solid dispersion of any one of claims 18-23, wherein the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is from 4:1 to 1:3.

25. The spray-dried solid dispersion of any one of claims 18-24, wherein the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is 4:1.

26. The spray-dried solid dispersion of any one of claims 18-24, wherein the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is 1:1.

27. The spray-dried solid dispersion of any one of claims 18-24, wherein the weight ratio of trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide to the pharmaceutically acceptable polymer is 3:7.

28. The spray-dried solid dispersion of any one of claims 18-27, further comprising a non-aqueous solvent.

29. The spray-dried solid dispersion of claim 28, wherein the non-aqueous solvent is selected from the group consisting of tert-butanol, n-propanol, n-butanol, isopropanol, ethanol, methanol, acetone, ethyl acetate, dimethyl carbonate, acetonitrile, dichloromethane, methyl ethyl ketone, methyl isobutyl ketone, 1-pentanol, methyl acetate, carbon tetrachloride, dimethyl sulfoxide, hexafluoroacetone, chlorobutanol, dimethyl sulfone, acetic acid, cyclohexane, and mixtures thereof.

30. The spray-dried solid dispersion of claim 28 or claim 29, wherein the non-aqueous solvent is selected from the group consisting of ethanol, methanol, propanol, butanol, isopropanol, tert-butanol, dichloromethane, and mixtures thereof.

31. The spray-dried solid dispersion of any one of claims 28-30, wherein the non-aqueous solvent is a mixture of dichloromethane and methanol.

32. The spray-dried solid dispersion of any one of claims 18-31, wherein trans-N-(3-(1-cyclopropyl-1H-pyrazol-4-yl)phenyl)-4-hydroxy-N-((trans-4-(4-methoxy-3-methylphenyl)cyclohexyl)methyl)cyclohexanecarboxamide is substantially amorphous.

33. A pharmaceutical formulation comprising a spray-dried solid dispersion of any one of claims 18-32 and optionally one or more pharmaceutical acceptable ingredients selected from the group consisting of one or more diluents, one or more disintegrants, one or more binders, one or more lubricants, one or more glidants, and one or more surfactants.

34. The pharmaceutical formulation of claim 33, wherein the one or more pharmaceutical acceptable ingredients are selected from the group consisting of microcrystalline cellulose, lactose monohydrate, croscarmellose sodium, magnesium stearate, colloidal silicon dioxide, mannitol, crospovidone, and sodium stearyl fumarate.

35. The pharmaceutical formulation of claim 34, wherein the one or more pharmaceutical acceptable ingredients are selected from the group consisting of microcrystalline cellulose, lactose monohydrate, croscarmellose sodium, magnesium stearate, and colloidal silicon dioxide.

36. The pharmaceutical formulation of any one of claims 33-35, wherein the pharmaceutical formulation is in tablet form.

37. The pharmaceutical formulation of any one of claims 33-35, wherein the pharmaceutical formulation is in capsule form.

38. A method of treating or preventing a liver disease or condition in a mammal, comprising administering to the mammal a crystalline form of any one of claims 1-15.

39. A method of treating or preventing a liver disease or condition in a mammal, comprising administering to the mammal a spray-dried solid dispersion of any one of claims 18-32.

40. The method of claim 38 or claim 39, wherein the liver disease or condition is an alcoholic or non-alcoholic liver disease or condition.

41. The method of claim 38 or claim 39, wherein the liver disease or condition is primary biliary cirrhosis, primary sclerosing cholangitis, cholestasis, nonalcoholic steatohepatitis (NASH), or nonalcoholic fatty liver disease (NAFLD).

42. The method of claim 40, wherein the alcoholic liver disease or condition is fatty liver (steatosis), cirrhosis, or alcoholic hepatitis.

43. The method of claim 40, wherein the non-alcoholic liver disease or condition is nonalcoholic steatohepatitis (NASH), or nonalcoholic fatty liver disease (NAFLD).

44. The method of claim 40, wherein the non-alcoholic liver disease or condition is nonalcoholic steatohepatitis (NASH).

45. The method of claim 40, wherein the non-alcoholic liver disease or condition is nonalcoholic steatohepatitis (NASH) and is accompanied by liver fibrosis.

46. The method of claim 40, wherein the non-alcoholic liver disease or condition is nonalcoholic steatohepatitis (NASH) without liver fibrosis.

47. The method of claim 40, wherein the non-alcoholic liver disease or condition is intrahepatic cholestasis or extrahepatic cholestasis.

48. A method of treating or preventing a liver fibrosis in a mammal, comprising administering to the mammal a crystalline form of any one of claims 1-15.

49. A method of treating or preventing a liver fibrosis in a mammal, comprising administering to the mammal a spray-dried solid dispersion of any one of claims 18-32.

50. The method of claim 48 or claim 49, wherein the mammal is diagnosed with hepatitis C virus (HCV), nonalcoholic steatohepatitis (NASH), primary sclerosing cholangitis (PSC), cirrhosis, Wilson's disease, hepatitis B virus (HBV), HIV associated steatohepatitis and cirrhosis, chronic viral hepatitis, non-alcoholic fatty liver disease (NAFLD), alcoholic steatohepatitis (ASH), nonalcoholic steatohepatitis (NASH), primary biliary cirrhosis (PBC), or biliary cirrhosis.

51. The method of claim 48 or claim 49, wherein the mammal is diagnosed with nonalcoholic steatohepatitis (NASH).

52. A method of treating or preventing a liver inflammation in a mammal, comprising administering to the mammal a crystalline form of any one of claims 1-15.

53. A method of treating or preventing a liver inflammation in a mammal, comprising administering to the mammal a spray-dried solid dispersion of any one of claims 18-32.

54. The method of claim 52 or claim 53, wherein the mammal is diagnosed with hepatitis C virus (HCV), nonalcoholic steatohepatitis (NASH), primary sclerosing cholangitis (PSC), cirrhosis, Wilson's disease, hepatitis B virus (HBV), HIV associated steatohepatitis and cirrhosis, chronic viral hepatitis, non-alcoholic fatty liver disease (NAFLD), alcoholic steatohepatitis (ASH), nonalcoholic steatohepatitis (NASH), primary biliary cirrhosis (PBC), or biliary cirrhosis.

55. The method of claim 52 or claim 53, wherein the mammal is diagnosed with nonalcoholic steatohepatitis (NASH).

56. The method of claim 52 or claim 53, wherein the liver inflammation is associated with inflammation in the gastrointestinal tract.

57. The method of claim 52 or claim 53, wherein the mammal is diagnosed with inflammatory bowel disease.

58. A method of treating or preventing a gastrointestinal disease or condition in a mammal, comprising administering to the mammal a compound of any one of claims 1-15, or a pharmaceutically acceptable salt or solvate thereof.

59. A method of treating or preventing a gastrointestinal disease or condition in a mammal, comprising administering to the mammal a spray-dried solid dispersion of any one of claims 18-32.

60. The method of claim 58 or claim 59, wherein the gastrointestinal disease or condition is necrotizing enterocolitis, gastritis, ulcerative colitis, Crohn's disease, inflammatory bowel disease, irritable bowel syndrome, gastroenteritis, radiation induced enteritis, pseudomembranous colitis, chemotherapy induced enteritis, gastro-esophageal reflux disease (GERD), peptic ulcer, non-ulcer dyspepsia (NUD), celiac disease, intestinal celiac disease, post-surgical inflammation, gastric carcinogenesis, graft versus host disease or any combination thereof.

61. The method of claim 58 or claim 59, wherein the gastrointestinal disease or condition is irritable bowel syndrome with diarrhea (IBS-D), irritable bowel syndrome with constipation (IBS-C), mixed IBS (IBS-M), unsubtyped IBS (IBS-U), or bile acid diarrhea (BAD).

62. A method of treating or preventing a disease or condition in a mammal that would benefit from treatment with a FXR agonist, comprising administering to the mammal a crystalline form of any one of claims 1-15.

63. A method of treating or preventing a disease or condition in a mammal that would benefit from treatment with a FXR agonist, comprising administering to the mammal a spray-dried solid dispersion of any one of claims 18-32.

64. The method of any one of claims 38-63, further comprising administering at least one additional therapeutic agent in addition to a crystalline form of any one of claims 1-15.

65. The method of any one of claims 38-63, further comprising administering at least one additional therapeutic agent in addition to a spray-dried solid dispersion of any one of claims 18-36.

66. A compound that is trans-N-(3-(1-Cyclopropyl-1H-pyrazol-4-yl)phenyl)-4-hydroxy-N-((trans-4-(4-methoxy-3-methylphenyl)cyclohexyl)methyl)cyclohexanecarboxamide, or a pharmaceutically acceptable salt, or solvate thereof, for use in medicine.
